(19) United States
(12) Patent Application Publication
(10) Pub. No.: US 2011/0160167 A1
(43) Pub. Date: Jun. 30, 2011

(54) Steroid Derivatives Acting As Glucocorticosteroid Receptor Agonists

(75) Inventors: Håkan Bladh, Lund (SE); Karl Edman, Molndal (SE); Thomas Hansson, Lund (SE); Karolina Lawitz, Lund (SE); Matti Lepistö, Lund (SE); Tesfaledet Mussie, Lund (SE)

(73) Assignee: AstraZeneca AB, Sodertalje (SE)

(21) Appl. No.: 12/809,131

(22) PCT Filed: Dec. 18, 2008
(86) PCT No.: PCT/SE2008/051489

§ 371 Date: Feb. 15, 2011

Related U.S. Application Data
(60) Provisional application No. 61/015,404, filed on Dec. 20, 2007.

(51) Int. Cl. A61K 31/573; C07J 53/00; A61K 31/58; A61P 11/00

(52) U.S. Cl. 514/170; 552/514; 514/174; 514/171

(57) ABSTRACT

The present invention provides compounds of formula (I) wherein n, p, R¹, R², X¹, X², X³, R^{3a}, R^{3b}, R⁴, R⁵ and R⁶ are as defined in the specification, a process for their preparation, pharmaceutical compositions containing them and their use in therapy.

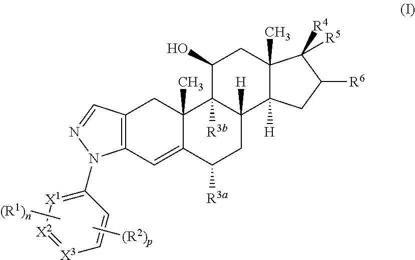

The present invention relates to compounds having glucocorticosteroid receptor agonist activity, processes for their preparation, pharmaceutical compositions containing them and their therapeutic use, particularly for the treatment of inflammatory and allergic conditions.

Glucocorticosteroids (GCs) that have anti-inflammatory properties are known and are widely used for the treatment of diseases such as inflammatory arthritides (e.g. rheumatoid arthritis, ankylosing spondylitis and psoriatic arthropathy), other rheumatoid diseases such as systemic lupus erythematosis, scleroderma, vascutitides including temporal arteritis and polyarteritis nodosa, inflammatory bowel disease such as Crohns disease and ulcerative colitis, lung diseases such as asthma and chronic obstructive airways disease, as well as many other conditions such as polymyalgia rheumatica. GCs have also been used very extensively for their immunosuppressive properties in the prevention and treatment of transplant rejection. Finally GCs have been used for their anti-tumour effects in a number of malignancies.

GCs act via specific glucocorticoid receptors (GR) that are members of the nuclear receptor superfamily. Ligand binding promotes receptor dimerisation, DNA binding, and transcriptional activation. This mechanism of GC action is well defined in vitro and is critical for regulation of the hypothalamic-pituitary-adrenal axis, gluconeogenesis as well as transcription of anti-inflammatory genes such as mitogen-activated protein kinase phosphatase-1 (MKP-1) and secretory leukocyte protease inhibitor (SLPI) in vivo. Ligand-bound receptor is also able to suppress gene transcription in a dimerisation-independent manner by interfering with the activity of transcription factors, such as AP-1 and NFkB, which are critically involved in the inflammatory reaction.

After ligand binding, the GR translocates from the cytoplasm of the cell to the nucleus and binds to glucocorticoid response elements in regulator regions of target genes. The activated GR then recruits co-factors, including the glucocorticoid receptor interacting protein 1 (GRIP-1) and steroid receptor co-activator 1 (SRC1). These accessory proteins bind to the receptor and link the GR with the general transcription machinery to drive transcription of target genes.

Glucocorticoid effects on transcription may be mediated by both the direct binding of activated GR to target DNA, homodimerisation and recruitment of co-activators (known as “transactivation”) but also by GR interfering with other transcription factor function, including AP-1 and NFkB, by complexing with these other transcription factors and preventing them from binding to their target genes leading to repression of the genes normally upregulated by AP-1 or NFkB (known as “transrepression”). These two modes of receptor activity are dissociable and negative effects on NFkB activity can be retained in the absence of transactivation. It appears that transrepression is largely responsible for mediating the therapeutically desirable anti-inflammatory activity of the GR. Interestingly, the IC₅₀ for inhibition of AP-1 or NFkB (0.04 nM) is lower than the EC₅₀ for activation of target genes (5 nM) and yet high doses of GCs are frequently required to treat patients with inflammatory disease. One explanation is that cytokines expressed at the site of inflammation may induce relative glucocorticoid resistance, for instance by activating AP-1 or NFkB. This is of importance as many pro-inflammatory cytokines signal by activation of NFkB and a major anti-inflammatory action of GCs is thought to be mediated by opposing NFkB action.

It has now surprisingly been found a new series of glucocorticosteroids having a long duration of action which have potential for once daily administration.

In accordance with the present invention, there is therefore provided a compound of formula

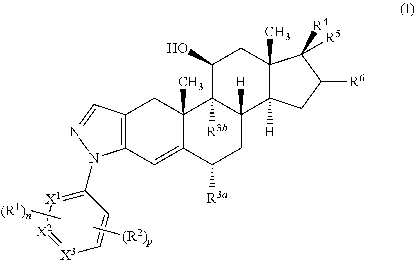

wherein
X¹, X² and X³ each represent CH or, alternatively, one of X¹, X² and X³ may additionally represent a nitrogen atom;
n and p each independently represent 0 or 1;
R¹ represents a halogen atom or a methyl or a methoxy group;
R² represents —CO₂CH₃, a halogen atom, or a methyl group optionally substituted by a hydroxyl or a —NR⁷R⁸ group;
R^{3a} represents a hydrogen atom and R^{3b} represents a hydrogen or fluorine atom;
R⁴ represents —C(O)CH₂OH or —C(O)—Y—CH₂R⁹;
R⁵ and R⁶ together with the carbon atoms to which they are attached form a 1,3-dioxolanyl group which is optionally substituted by at least one substituent selected from C₁-C₃ alkyl and C₃-C₈ cycloalkyl;
R⁷ and R⁸ each independently represent a hydrogen atom, or a C₁-C₃ alkyl or a C₁-C₃ hydroxyalkyl group, or
R⁷ and R⁸ together with the nitrogen atom to which they are attached form a 3- to 8-membered saturated or partially saturated heterocyclic ring optionally containing a further ring heterogroup selected from nitrogen, S(O)ₘ and oxygen, the heterocyclic ring being optionally substituted by at least one substituent selected from hydroxyl, C₁-C₃ alkyl and C₁-C₃ hydroxyalkyl;
m is 0, 1 or 2;
Y represents an oxygen or sulphur atom or a group >NH; and
R⁹ represents a hydrogen or a halogen atom or a methyl or a cyano group; or a pharmaceutically acceptable salt thereof.

In the context of the present specification, unless otherwise stated, an alkyl substituent group or an alkyl moiety in a substituent group may be linear or branched. Examples of C₁-C₆ alkyl groups/moieties include methyl, ethyl, propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, tert-butyl, n-pentyl, isopentyl, neopentyl and n-hexyl. Similarly, an alkylene group/moiety may be linear or branched. Examples of C₁-C₆ alkylene groups/moieties include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. A C₁-C₆ hydroxyalkyl substituent group/moiety will comprise at least one hydroxyl group, e.g. one, two, three or four hydroxyl groups, examples of which include —CH₂OH, —CH₂CH₂OH, —CH₂CH₂CH₂OH and —CH(CH₂OH)₂. For the avoidance of doubt, it should be understood that when R⁷ and R⁸ are defined as representing a heterocyclic ring, the definition is not intended to include unstable structures or any O—O, O—S or S—S bonds and that a substituent, if present, may be attached to any suitable ring atom.

When any chemical moiety or group in formula (I) is described as being optionally substituted, it will be appreciated that the moiety or group may be either unsubstituted or substituted by one or more of the specified substituents. It will be appreciated that the number and nature of substituents will be selected so as to avoid sterically undesirable combinations.

In formula (I), X¹, X² and X³ each represent CH (so as to form a phenyl ring) or, alternatively, one of X¹, X² and X³ may additionally represent a nitrogen atom (so as to form a pyridyl ring).

In an embodiment of the invention, X¹, X² and X³ each represent CH.

In an embodiment of the invention n is 1 and p is 0 or 1.

R¹ represents a halogen atom (e.g. fluorine, chlorine, bromine or iodine) or a methyl or a methoxy group.

In an embodiment of the invention, R¹ represents a fluorine atom.

In another embodiment, when n is 1 and X³ represents CH, X³ is substituted by R¹.

R² represents —CO₂CH₃, a halogen atom (e.g. fluorine, chlorine, bromine or iodine) or a methyl group optionally substituted by a hydroxyl or a —NR⁷R⁸ group.

In one embodiment, R² represents —CO₂CH₃.

In another embodiment, R² represents a methyl group substituted by one hydroxyl group, i.e. —CH₂OH.

In a further embodiment, R² represents a methyl group substituted by one —NR⁷R⁸ group, i.e. —CH₂NR⁷R⁸.

In a still further embodiment, R² represents a methyl group substituted by two hydroxyl groups or two —NR⁷R⁸ groups or by one hydroxyl group and one —NR⁷R⁸ group.

In an embodiment of the invention R^{3a} and R^{3b} each represent a hydrogen atom.

R⁴ represents —C(O)CH₂OH or —C(O)—Y—CH₂R⁹.

In an embodiment of the invention, R⁴ represents —C(O)CH₂OH.

In another embodiment, R⁴ represents —C(O)—Y—CH₂R⁹ where Y represents an oxygen or sulphur atom and R⁹ represents a hydrogen or a halogen (e.g. fluorine, chlorine, bromine or iodine) atom or a methyl or a cyano group.

In yet another embodiment, R⁴ represents —C(O)—Y—CH₂R⁹ where Y represents an oxygen or sulphur atom and R⁹ represents a fluorine atom or a methyl or a cyano group. In a further aspect, Y represents an oxygen or, particularly, sulphur atom and R⁹ represents a cyano group.

R⁵ and R⁶ together with the carbon atoms to which they are attached form a 1,3-dioxolanyl group which is optionally substituted by at least one substituent (particularly one or two substituents independently) selected from C₁-C₃ alkyl (methyl, ethyl, n-propyl or iso-propyl) and C₃-C₈ cycloalkyl (e.g. cyclopropyl, cyclobutyl, cyclopentyl or cyclohexyl).

In one aspect, R⁵ and R⁶ together with the carbon atoms to which they are attached form a 1,3-dioxolanyl group which is substituted by one or two C₁-C₃ alkyl groups.

In another aspect, R⁵ and R⁶ together with the carbon atoms to which they are attached form a 1,3-dioxolanyl group which is substituted by one or two methyl or n-propyl groups.

In still another aspect, R⁵ and R⁶ together with the carbon atoms to which they are attached form a 1,3-dioxolanyl group which is substituted by a C₃-C₈, preferably C₃-C₆, cycloalkyl group, in particular a cyclohexyl group.

The substituent(s) in the 1,3-dioxolanyl group is/are preferably attached at the 2-position, i.e., attached to the carbon atom between the two ring oxygen atoms.

R⁷ and R⁸ each independently represent a hydrogen atom, or a C₁-C₃ alkyl (methyl, ethyl, n-propyl or isopropyl) or a C₁-C₃ hydroxyalkyl (e.g. hydroxymethyl, —(CH₂)₂OH, —(CH₂)₃OH or —CH(CH₂OH)₂) group, or R⁷ and R⁸ together with the nitrogen atom to which they are attached form a 3- to 8-membered, preferably 5- to 6-membered, saturated or partially saturated heterocyclic ring optionally containing a further ring heterogroup selected from nitrogen, S(O)ₘ and oxygen, the heterocyclic ring being optionally substituted by at least one substituent, e.g. one, two, three or four substituents independently, selected from hydroxyl, C₁-C₃ alkyl (methyl, ethyl, n-propyl or isopropyl) and C₁-C₃ hydroxyalkyl (e.g. hydroxymethyl, —(CH₂)₂OH, —(CH₂)₃OH or —CH(CH₂OH)₂).

Examples of 3- to 8-membered saturated or partially saturated heterocyclic rings include morpholine, azetidine, pyrrolidine, piperidine, piperazine, 3-pyrroline, isoindoline, tetrahydroquinoline and thiomorpholine.

In one embodiment, R⁷ and R⁸ each independently represent a hydrogen atom or a C₁-C₂ alkyl (particularly ethyl) or C₂-C₃ hydroxyalkyl group.

In another embodiment, R⁷ and R⁸ together with the nitrogen atom to which they are attached form a 5- to 6-membered saturated or partially saturated heterocyclic ring optionally containing a further ring heterogroup selected from nitrogen, S(O)ₘ and oxygen, the heterocyclic ring being optionally substituted by one or two substituents independently selected from hydroxyl, C₁-C₃ alkyl and C₁-C₃ hydroxyalkyl.

In yet another embodiment, R⁷ and R⁸ together with the nitrogen atom to which they are attached form a 5- to 6-membered saturated heterocyclic ring optionally containing a further ring heterogroup selected from nitrogen, sulphur and oxygen (e.g. pyrrolidinyl, piperidinyl, piperazinyl, thiomorpholinyl or morpholinyl), the heterocyclic ring being optionally substituted by one or two substituents independently selected from hydroxyl, C₁-C₃ alkyl and C₁-C₃ hydroxyalkyl.

In a still further embodiment, R⁷ and R⁸ together with the nitrogen atom to which they are attached form a 5- to 6-membered saturated heterocyclic ring optionally containing a further ring heterogroup selected from nitrogen, sulphur and oxygen, the heterocyclic ring being optionally substituted by one or two substituents independently selected from hydroxyl, methyl and hydroxymethyl.

In an embodiment of the invention, m is 0.

In an embodiment of the invention, Y represents a sulphur atom.

In an embodiment of the invention, R⁹ represents a fluorine atom or a methyl or a cyano group.

In an embodiment of the invention,
X¹, X² and X³ each represent CH;
n is 1;
p is 0 or 1;
R¹ represents a fluorine atom;
R² represents —CO₂CH₃, or a methyl group optionally substituted by a hydroxyl or a —NR⁷R⁸ group;
R^{3a} and R^{3b} each represent a hydrogen atom;
R⁴ represents —C(O)CH₂OH or —C(O)—S—CH₂CN;
R⁵ and R⁶ together with the carbon atoms to which they are attached form a 1,3-dioxolanyl group which is optionally substituted by at least one substituent selected from C₁-C₃ alkyl and cyclohexyl;
R⁷ and R⁸ each independently represent a hydrogen atom, or a C₁-C₃ alkyl or a C₁-C₃ hydroxyalkyl group, or
R⁷ and R⁸ together with the nitrogen atom to which they are attached form a 5- to 6-membered saturated heterocyclic ring optionally containing a further ring heterogroup selected from nitrogen, S(O)ₘ and oxygen, the heterocyclic ring being optionally substituted by at least one substituent selected from hydroxyl, methyl and hydroxymethyl; and
m is 0.

Examples of compounds of the invention include:
1-[(4aR,4bS,5S,6aS,6bS,8R,9aR,10aS,10bS)-1-[4-fluoro-3-(morpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-(4aR,4bS,5S,6aS,6bS,8S,9aR,10aS,10bS)-1-[4-fluoro-3-(morpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-fluoro-3-(pyrrolidin-1-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{4-fluoro-3-[(4-methylpiperazin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-{4-fluoro-3-[(4-methylpiperazin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{3-[(diethylamino)methyl]-4-fluorophenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-fluoro-3-(thiomorpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-[4-fluoro-3-(thiomorpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{4-fluoro-3-[(4-hydroxypiperidin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-{4-fluoro-3-[(4-hydroxypiperidin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[(3-hydroxypropyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-fluoro-3-{[(3-hydroxypropyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[4-(hydroxymethyl)piperidin-1-yl]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-fluoro-3-{[4-(hydroxymethyl)piperidin-1-yl]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[(2-hydroxyethyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo[3′,4′]-cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone;
1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone;
1-[(4aR,4bS,5S,6aS,6bS,9aR,10aS,10bS)-1-[4-Fluoro-3-(hydroxymethyl)phenyl]-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
Methyl 2-fluoro-5-[(4aR,4bS,5S,6aS,6bS,8R,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]benzoate;
Methyl 2-fluoro-5-[(4aR,4bS,5S,6aS,6bS,8S,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]benzoate;
1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b-decahydro[1,3]dioxolo[3′,4′]cyclopenta-[1′,2′:5,6]naphtho[1,2-f]indazol-6b(1H)-yl]-2-hydroxyethanone;
(4aR,4bS,5S,6aS,6bS,8R,9aR,10aS,10bS)-6b-(cyanomethylthiocarbonyl)-1-(4-fluorophenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol;
(4aR,4bS,5S,6aS,6bS,9aR,10aS,10bS)-6b-(cyanomethylthiocarbonyl)-1-(4-fluorophenyl)-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol;
(4aR,4bS,5S,6aS,6bS,8R,9aR,10aS,10bS)-8-cyclohexyl-6b-(cyanomethylthiocarbonyl)-1-(4-fluorophenyl)-5-hydroxy-4a,6a-dimethyl-4,4a,4b,5,6,6a,9a,10,10a,10b,11,12-dodecahydro[1,3]dioxolo[3′,4′]cyclopenta-[1′,2′:5,6]naphtho[1,2-f]indazol;
   and pharmaceutically acceptable salts of any one thereof.

It should be noted that each of the chemical compounds listed above represents a particular and independent aspect of the invention.

The present invention further provides a process for the preparation of a compound of formula (I) or a pharmaceutically acceptable salt thereof as defined above which comprises reacting a compound of formula (II)

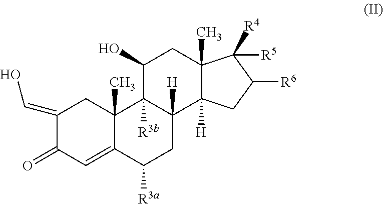

wherein R^{3a}, R^{3b}, R⁴, R⁵ and R⁶ are as defined in formula (I), with a compound of formula (III) or an acid addition salt (e.g. hydrochloride salt) thereof

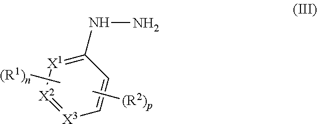

wherein n, p, R¹, R², X¹, X² and X³ are as defined in formula (I), and optionally thereafter carrying out one or more of the following procedures:
converting a compound of formula (I) into another compound of formula (I)
removing any protecting groups
forming a pharmaceutically acceptable salt.

The above process is conveniently carried out in the presence of an organic solvent such as acetic acid/water mixture at room temperature (20° C.) or, alternatively, in the presence of an organic solvent such as ethanol at a temperature in the range from room temperature (20° C.) to 90° C. Preferably, the reaction is carried out in the presence of a base, e.g. an alkali metal acetate such as potassium acetate.

The compounds of formula (II) may be prepared by reacting a compound of formula (IV)

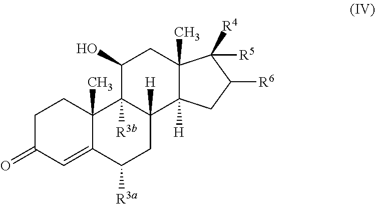

wherein R^{3a}, R^{3b}, R⁴, R⁵ and R⁶ are as defined in formula (II), with methyl or ethyl formate in the presence of a base such as sodium hydride, in a manner analogous to the method described in the journal article by Wuest, F et al., Steroids, 68 (2003), 177-191.

Compounds of formulae (III) and (IV) are either commercially available, are well known in the literature or may be prepared easily using known techniques.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate.

The compounds of formula (I) and pharmaceutically acceptable salts thereof may exist in solvated, for example hydrated, as well as unsolvated forms, and the present invention encompasses all such solvated forms.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of glucocorticoid receptor activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

Thus, the present invention provides a compound of formula (I) or a pharmaceutically acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention (including pharmaceutically acceptable salts) may be used in the treatment of asthma {such as bronchial, allergic, intrinsic, extrinsic or dust asthma, particularly chronic or inveterate asthma (for example late asthma or airways hyper-responsiveness)}, chronic obstructive pulmonary disease (COPD) or allergic rhinitis.

The invention also provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the invention (that is, compounds of formula (I) and pharmaceutically acceptable salts thereof) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention (that is, compounds of formula (I) and pharmaceutically acceptable salts thereof) may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention and a beta-adrenoreceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention and a chromone, such as sodium cromoglycate or nedocromil sodium.

The present invention still further relates to the combination of a compound of the invention with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a glucocorticoid receptor agonist.

In a further aspect the present invention provides a combination (for example for the treatment of COPD, asthma or allergic rhinitis) of a compound of formula (I) or a pharmaceutically acceptable salt thereof as hereinbefore defined and one or more agents independently selected from:
a non-steroidal glucocorticoid receptor (GR-receptor) agonist;
a selective β₂ adrenoceptor agonist (such as metaproterenol, isoproterenol, isoprenaline, albuterol, salbutamol, formoterol, salmeterol, terbutaline, orciprenaline, bitolterol mesylate, pirbuterol or indacaterol);
a phosphodiesterase inhibitor (such as a PDE4 inhibitor);
a protease inhibitor (such as a neutrophil elastase or matrix metalloprotease MMP-12 inhibitor);
a glucocorticoid;
an anticholinergic agent;
a modulator of chemokine receptor function (such as a CCR1 receptor antagonist); and
an inhibitor of kinase function (such as the kinases p38 or IKK).

The invention also provides a pharmaceutical product comprising, in combination, a preparation of a first active ingredient which is a compound of formula (I) or a pharmaceutically acceptable salt thereof as hereinbefore defined, and a preparation of a second active ingredient which is
a non-steroidal glucocorticoid receptor (GR-receptor) agonist;
a selective β₂ adrenoceptor agonist;
a phosphodiesterase inhibitor;
a protease inhibitor;
a glucocorticoid;
an anticholinergic agent;
a modulator of chemokine receptor function; or
an inhibitor of kinase function;
   for simultaneous, sequential or separate use in therapy.

In another aspect, the invention provides a kit comprising a preparation of a first active ingredient which is a compound of formula (I) or a pharmaceutically acceptable salt thereof as hereinbefore defined, and a preparation of a second active ingredient which is
a non-steroidal glucocorticoid receptor (GR-receptor) agonist;
a selective β₂ adrenoceptor agonist;
a phosphodiesterase inhibitor;
a protease inhibitor;
a glucocorticoid;
an anticholinergic agent;
a modulator of chemokine receptor function; or
an inhibitor of kinase function;
   and instructions for the simultaneous, sequential or separate administration of the preparations to a patient in need thereof.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples in which the following abbreviations are used:
EtOAc ethyl acetate
CH₂Cl₂ dichloromethane
DMF N,N-dimethylformamide
NaH sodium hydride
MgSO₄ magnesium sulphate
NaNO₂ sodium nitrite
SnCl₂ tin (II) chloride
NaOH sodium hydroxide
Na₂SO₄ sodium sulphate
DIEA diisopropylethylamine
DMSO dimethylsulfoxide
THF tetrahydrofuran
TFA trifluoroacetic acid
HCl hydrochloric acid
NaHCO₃ sodium hydrogen carbonate
Et₃N triethylamine
MeCN acetonitrile
EDTA ethylenediaminetetraacetic acid
conc. concentrated
rt room temperature
h hours
min minutes
M molar
MS mass spectrometry
APCI atmospheric chemical ionisation method
ESI electron spray ionisation method
NMR nuclear magnetic resonance
SCX Solid phase extraction with a sulfonic acid sorbent
HPLC High performance liquid chromatography

### General Methods

NMR spectra were recorded on a Varian Mercury-VX 300 MHz instrument or a Varian Inova 400 MHz instrument. The central peaks of chloroform-d (H 7.26 ppm), acetone-d₆ (H 2.05 ppm), acetonitrile-d₃ (δ_{H} 1.94 ppm) or DMSO-d₆ (H 2.50 ppm) were used as internal references.

The following method was used for LC/MS analysis:

Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

Column chromatography was carried out using silica gel (0.040-0.063 mm, Merck). For preparative HPLC either a Kromasil KR-100-5-C18 column (250×20 mm, Akzo Nobel) and mixtures of acetonitrile/water (0.1% TFA) at a flow rate of 10 ml/min or a XTerra® Prep MS C₁₈ OBD™ Column, 5 μm, 19×50 mm (acetonitrile/water/0.1% NH₃) at a flow rate of 20 ml/min was used. UV=254 nm or 220 nm was used for detection.

Unless stated otherwise, starting materials were commercially available. All solvents and commercial reagents were of laboratory grade and were used as received.

### Intermediate 1

### (4aR,4bS,5S,6aS,6bS,9aR,10aS,10bS)-6b-Glycoloyl-5-hydroxy-4a,6a-dimethyl-8-propyl-3,4,4a,4b,5,6,6a,6b,9a,10,10a,10b,11,12-tetradecahydro-2H-naphtho[2′,1′:4,5]indeno[1,2-d][1,3]-dioxol-2-one

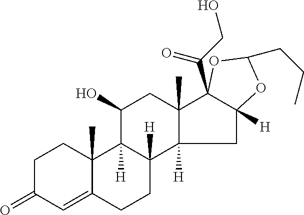

(4aR,4bS,5S,6aS,6bS,9aR,10aS,10bS)-6b-Glycoloyl-5-hydroxy-4a,6a-dimethyl-8-propyl-4a,4b,5,6,6a,6b,9a,10,10a,10b,11,12-dodecahydro-2H-naphtho[2′,1′:4,5]indeno[1,2-d][1,3]dioxol-2-one (budesonide) (7 g, 16.3 mmol) was dissolved in a mixture of 200 ml ethanol and 100 ml toluene. Tris(triphenylphosphine)rhodium(I) chloride (2.1 g, 2.3 mmol) was added and the mixture was stirred under a hydrogen atmosphere for 40 h. After completion, monitored by LC-MS, the solvent was evaporated and the residue was redissolved in CH₂Cl₂. Heptane was added and the mixture was stirred for 1 h. The precipitate was filtered off and washed with CH₂Cl₂/heptane. The crude product was used directly in the next step. APCI-MS m/z: 433 [MH⁺].

### Intermediate 2

### (4aR,4bS,5S,6aS,6bS,9aR,10aS,10bS)-6b-({[tert-Butyl(dimethyl)silyl]oxy}acetyl)-5-hydroxy-4a,6a-dimethyl-8-propyl-3,4,4a,4b,5,6,6a,6b,9a,10,10a,10b,11,12-tetradecahydro-2H-naphtho[2′,1′:4,5]indeno[1,2-d][1,3]dioxol-2-one

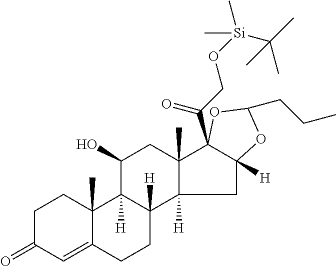

The crude product of intermediate 1 (7.03 g, 16.3 mmol) was dissolved in a mixture of CH₂Cl₂/pyridine (2:1, 90 ml) and tert-butyldimethylchlorosilane (9.7 ml, 61.8 mmol) was added. The mixture was stirred over night. The solvent was evaporated and the crude product was purified on silica (EtOAc/heptane, gradient from 10 to 70% EtOAc) to give the title compound (7.3 g, 82%). APCI-MS m/z: 547 [MH⁺].

### Intermediate 3

### (4aR,4bS,5S,6aS,6bS,9aR,10aS,10bS)-6b-({[tert-Butyl(dimethyl)silyl]oxy}acetyl)-5-hydroxy-4a,6a-dimethyl-2-oxo-8-propyl-3,4,4a,4b,5,6,6a,6b,9a,10,10a,10b,11,12-tetradecahydro-2H-naphtho[2′,1′:4,5]indeno[1,2-d][1,3]dioxole-3-carbaldehyde

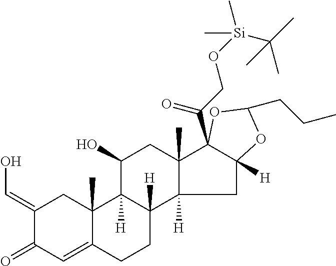

Intermediate 2 (1 g, 1.8 mmol) dissolved in toluene (20 ml) were mixed under argon in a dry vessel with 55% NaH in oil (1.5 g, 36.6 mmol). Ethyl formate (2.2 ml, 27.5 mmol) was added. After the hydrogen evolution had ceased it was heated under microwave irradiation at 45° C. for 60 minutes. The mixture was partitioned between water and EtOAc. The organic phase was washed with brine, dried with MgSO₄ and evaporated. The crude product contained mostly product and was used in the next step without further purification.

APCI-MS m/z: 575 [MH⁺].

### Intermediate 4

### (2-Fluoro-5-hydrazinylphenyl)methanol hydrochloride

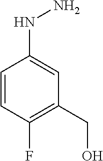

(5-Amino-2-fluorophenyl)methanol (3.6 g, 25.8 mmol) was dissolved in conc. HCl (39 ml) and cooled to 0° C. A solution of NaNO₂ (1.89 g, 27.4 mmol) in water (9.4 ml) was added dropwise during 20 minutes at 0° C. The mixture was stirred for 15 minutes and a solution of SnCl₂ (11 g, 57.4 mmol) in conc. HCl (11.5 ml) was added dropwise. The mixture was allowed to stir for 60 minutes at 0° C. and basified by addition of aqueous 14N NaOH and extracted with EtOAc. The organic phase was washed with brine and extracted with a solution of HCl (1 M). The water phase was freeze-dried to give the title compound (1.65 g, 33%). APCI-MS m/z: 157 [MH⁺].

### Intermediate 5

### 2-{[tert-Butyl(dimethyl)silyl]oxy}-1-[(4aR,4bS,5S,6aS,6bS,9aR,10aS,10bS)-1-[4-fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone

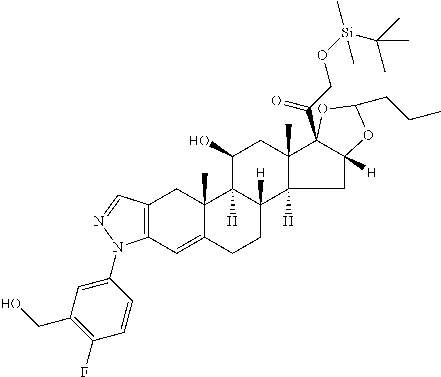

Intermediate 3 (1.05 g, 1.83 mmol) was dissolved in ethanol (10 ml). (2-Fluoro-5-hydrazinylphenyl)methanol hydrochloride (0.42 g, 2.20 mmol) and potassium acetate (0.23 g, 2.29 mmol) were added and the mixture was heated under microwave irradiation at 90° C. for 10 minutes. The crude product was purified on silica (EtOAc/heptane 1:3) to give the title compound (1.2 g, 92%). APCI-MS m/z: 695 [MH⁺].

### Intermediate 6

### 5-[(4aR,4bS,5S,6aS,6bS,9aR,10aS,10bS)-6b-({[tert-butyl(dimethyl)silyl]oxy}acetyl)-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]-2-fluorobenzyl methanesulfonate

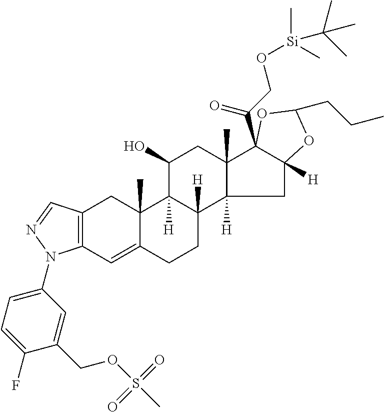

Intermediate 5 (1.29 g, 1.86 mmol), Et₃N (0.28 ml, 2.04 mmol) and methanesulfonyl chloride were stirred in CH₂Cl₂ (20 ml) overnight. The mixture was partitioned between CH₂Cl₂ and brine and the organic phase was dried over MgSO₄ and evaporated to give the crude title compound (1 g, 70%) which was used in the next step without further purification. APCI-MS m/z: 773 [MH⁺].

### EXAMPLE 1

### 1-[(4aR,4bS,5S,6aS,6bS,8R,9aR,10aS,10bS)-1-[4-fluoro-3-(morpholin-4-ylmethyl)phenyl]-5-hydroxy-4a,6a-dimethyl-8-propyl-4,4a,4b,5,6,6a,9a,10,10a,10b,11,12-dodecahydro[1,3]-dioxolo[3′,4′]cyclopenta-[1′,2′:5,6]naphtho[1,2-f]indazol-6b(1H)-yl]-2-hydroxyethanone trifluoroacetic acid

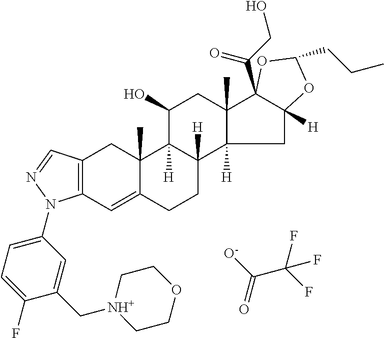

Intermediate 6 (200 mg, 0.26 mmol) was dissolved in DMF (4 ml) and morpholine (100 mg, 1.15 mmol) was added. The mixture was stirred for 2 h. TFA/water (1:1) was added until the protection group was removed. The sample was purified on preparative HPLC (MeCN/water/0.1% TFA) to give the title compound (43 mg, 25%).

¹H NMR (400 MHz, CD₃CN) δ 7.72 (1H, dd); 7.62-7.58 (1H, m); 7.39 (1H, s); 7.32 (1H, t); 6.17 (1H, d); 4.83 (1H, d); 4.60 (1H, t); 4.49 (1H, d); 4.45-4.42 (1H, m); 4.29-4.22 (3H, m); 3.84 (4H, bs); 3.17 (4H, bs); 2.96 (1H, d); 2.66 (1H, d); 2.48 (1H, t); 2.00 (1H, dd); *(2H in solvent peaks) 1.78-1.57 (6H, m); 1.46-1.37 (2H, m); 1.24 (3H, s); 1.19 (1H, dd); 1.09-0.98 (1H, m); 0.91 (3H, t); 0.86 (3H, s). APCI-MS m/z: 650 [MH⁺].

### EXAMPLES 2 TO 15

The following compounds were synthesised by methods analogous to that described in Example 1.

| | | | | APCI |
|---|---|---|---|---|
| Structure | Ex | Compound | ¹H NMR | m/z |
| 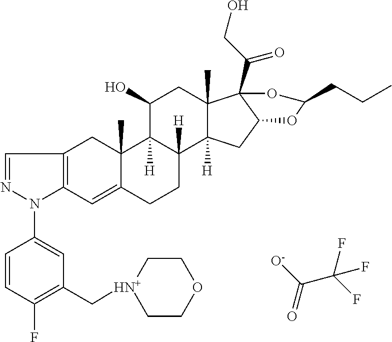 | 2 | 1- (4aR,4bS,5S,6aS,6bS, 8S,9aR,10aS,10bS)- 1-[4-fluoro-3- (morpholin-4- ylmethyl)phenyl]-5- hydroxy-4a,6a- dimethyl-8- propyl- 4,4a,4b,5,6,6a,9a,10, 10a,10b,11,12- dodecahydro[1,3]dioxolo [3′,4′]cyclopenta [1′,2′:5,6]naphtho[1,2-f]indazol- 6b(1H)-yl]- 2-hydroxyethanone trifluoroacetic acid | ¹HNMR (400 MHz, CD₃CN) δ 7.71 (1 H, d); 7.60-7.55 (1 H, m); 7.38 (1 H, s); 7.31 (1 H, t); 6.16 (1 H, s); 5.23 (1 H, t); 5.14 (1 H, d); 4.58 (1 H, d); 4.44-4.40 (1 H, m); 4.25-4.15 (3 H, m); 3.83 (4 H, bs); 3.10 (4 H, bs); 2.96 (1 H, d); 2.65 (1 H, d); 2.53-2.42 (1 H, m); 1.80-1.65 (4 H, m); 1.50- 1.43 (2 H, m); 1.38-1.29 (2 H, m); 1.24 (3 H, s); 1.21 (1 H, dd); 1.13- 1.03 (1 H, m); 0.92-0.88 (6 H, m). | 650 |
| 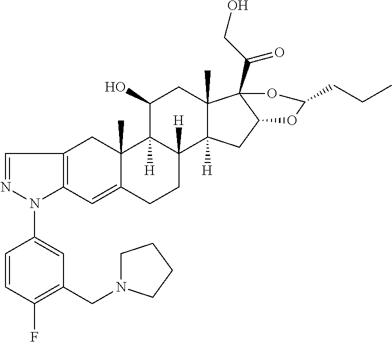 | 3 | 1- [(4aR,4bS,5S,6aS,6bS, 8R,9aR,10aS,10bS)- 1-[4-fluoro-3- (pyrrolidin-1- ylmethyl)phenyl]-5- hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone | ¹H NMR (400 MHz, CDCl₃) δ 7.93 (1 H, bs); 7.71 (1 H, bs); 7.42 (1 H, s); 6.60 (1 H, s); 4.92 (1 H, d); 4.60-4.49 (3 H, m); 4.33- 4.25 (2 H, m); 3.74 (1 H, s); 3.04-2.79 (3 H, m); 2.74- 2.58 (2 H, m); 2.53- 2.42 (1 H, m); 2.37-1.62 (13 H, m); 1.51-1.42 (3 H, m); 1.33-1.24 (4 H, m); 1.19-1.06 (2 H, m); 1.00-0.88 (6 H, m). | 634 |
| 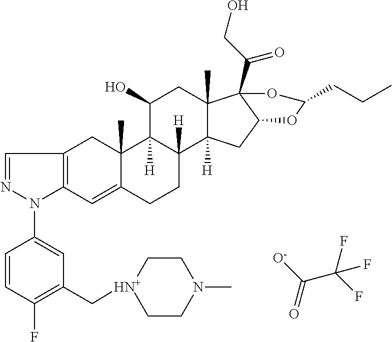 | 4 | 1- [(4aR,4bS,5S,6aS,6bS, 8R,9aR,10aS,10bS)- 1-{4-fluoro-3-[(4- methylpiperazin-1- yl)methyl]phenyl}-5- hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (400 MHz, CD₃CN) δ 7.53 (1 H, dd); 7.45-7.40 (1 H, m); 7.39 (1 H, s); 7.23 (1 H, t); 6.12 (1 H, s); 4.85 (1 H, d); 4.61 (1 H, t); 4.50 (1 H, d); 4.47-4.44 (1 H, m); 4.25 (1 H, d); 3.74 (2 H, s); 3.40 (2 H, bs); 3.07-2.91 (4 H, m); 2.75 (3 H, s); 2.68 (1 H, d); 2.50 (1 H, t); 2.29 (1 H, d); 1.80-1.58 (6 H, m); 1.47-1.38 (2 H, m); 1.27 (3 H, s); 1.21 (1 H, dd); 1.10-0.99 (1 H, m); 0.92 (3 H, t); 0.87 (3 H, s). | 663 |
| 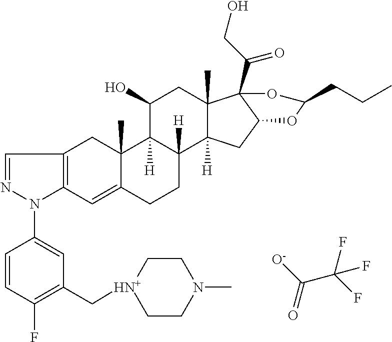 | 5 | 1- [(4aR,4bS,5S,6aS,6bS, 8S,9aR,10aS,10bS)- 1-{4-fluoro-3-[(4- methylpiperazin-1- yl)methyl]phenyl}-5- hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.51 (1 H, dd); 7.44-7.38 (1 H, m); 7.37 (1 H, s); 7.22 (1 H, t); 6.11 (1 H, d); 5.23 (1 H, t); 5.14 (1 H, d); 4.58 (1 H, d); 4.45-4.41 (1 H, m); 4.18 (1 H, d); 3.71 (2 H, s); 3.37 (2 H, bs); 3.03-2.87 (4 H, m); 2.73 (3 H, s); 2.66 (1 H, d); 1.87-1.61 (5 H, m); 1.51-1.28 (4 H, m); 1.25 (3 H, s); 1.21 (1 H, dd); 1.14-1.01 (1 H, m); 0.93-0.87 (6 H, m). | 663 |
| 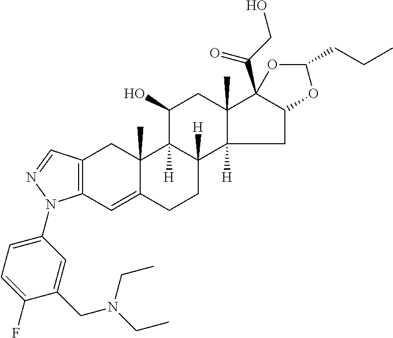 | 6 | 1- [(4aR,4bS,5S,6aS,6bS, 8R,9aR,10aS,10bS)- 1-{3-[(diethylamino) methyl]-4- fluorophenyl}-5- hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone | ¹H NMR (400 MHz, acetone-d₆) δ 7.94-7.84(1 H, m); 7.69- 7.59 (1 H, m); 7.42- 7.34 (2 H, m); 6.26-6.24 (1 H, m); 4.86 (1 H, d); 4.62 (1 H, t); 4.57-4.49 (3 H, m); 4.26 (1 H, d); 3.63-3.51 (4 H, m); 3.03 (2 H, d); 2.57-2.46 (1 H, m); 2.37-2.30 (1 H, m); 1.79-1.59 (5 H, m); 1.50-1.20 (12 H, m); 1.11-1.00 (1 H, m); 0.94-0.89 (6 H, m). | 636 |
| 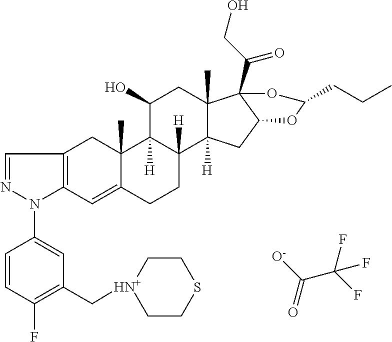 | 7 | 1- [(4aR,4bS,5S,6aS,6bS, 8R,9aR,10aS,10bS)- 1-[4-fluoro-3- (thiomorpholin-4- ylmethyl)phenyl]- 5-hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.71 (1 H, dd); 7.61-7.55 (1 H, m); 7.38 (1 H, s); 7.31 (1 H, t); 6.16 (1 H, d); 4.83 (1 H, d); 4.60 (1 H, t); 4.49 (1 H, d); 4.45-4.41 (1 H, m); 4.28-4.19 (3 H, m); 3.31 (4 H, bs); 2.99-2.81 (5 H, m); 2.66 (1 H, d); 1.79- 1.56 (6 H, m); 1.50-1.34 (2 H, m); 1.24 (3 H, s); 1.19 (1 H, dd); 1.11-0.96 (1 H, m); 0.91 (3 H, t); 0.86 (3 H, s). | 666 |
| 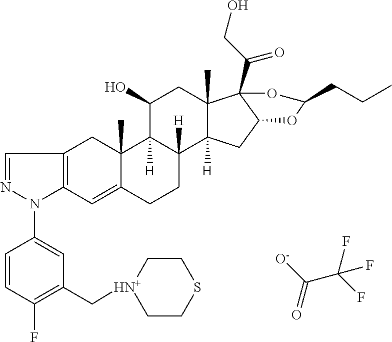 | 8 | 1- [(4aR,4bS,5S,6aS,6bS, 8S,9aR,10aS,10bS)- 1-[4-fluoro-3- (thiomorpholin-4- ylmethyl)phenyl]- 5-hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.72 (1 H, dd); 7.62-7.56 (1 H, m); 7.38 (1 H, s); 7.32 (1 H, t); 6.15 (1 H, d); 5.23 (1 H, t); 5.14 (1 H, d); 4.58 (1 H, d); 4.44-4.40 (1 H, m); 4.28 (2 H, s); 4.18 (1 H, d); 3.36 (4 H, bs); 2.98-2.86 (5 H, m); 2.65 (1 H, d); 1.88-1.61 (5 H, m); 1.51- 1.26 (4 H, m); 1.25- 1.18 (4 H, m); 1.16-1.01 (1 H, m); 0.94-0.86 (6 H, m). | 666 |
| 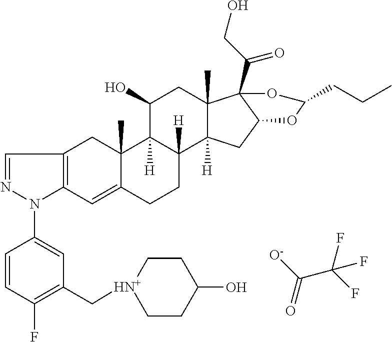 | 9 | 1- [(4aR,4bS,5S,6aS,6bS, 8R,9aR,10aS,10bS)- 1-{4-fluoro-3-[(4- hydroxypiperidin-1- yl)methyl]phenyl}- 5-hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (400 MHz, CD₃CN) δ 7.74 (1 H, dd); 7.63-7.59 (1 H, m); 7.39 (1 H, s); 7.33 (1 H, t); 6.16 (1 H, d); 4.83 (1 H, d); 4.60 (1 H, t); 4.49 (1 H, d); 4.45-4.42 (1 H, m); 4.31 (2 H, s); 4.24 (1 H, d); 3.98 (1 H, bs); 3.28 (2 H, bs); 2.96 (1 H, d); 2.66 (1 H, d); 2.48 (1 H, t); 2.04-1.97 (2 H, m); 1.82- 1.54 (8 H, m); 1.47-1.36 (2 H, m); 1.24 (3 H, s); 1.19 (1 H, dd); 1.09-0.97 (1 H, m); 0.91 (3 H, t); 0.86 (3 H, s). | 664 |
| 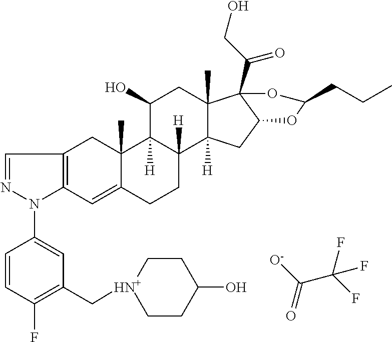 | 10 | 1- [(4aR,4bS,5S,6aS,6bS, 8S,9aR,10aS,10bS)- 1-{4-fluoro-3-[(4- hydroxypiperidin-1- yl)methyl]phenyl}- 5-hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.74 (1 H, dd); 7.63-7.57 (1 H, m); 7.39 (1 H, s); 7.32 (1 H, t); 6.16 (1 H, d); 5.23 (1 H, t); 5.14 (1 H, d); 4.58 (1 H, d); 4.44-4.40 (1 H, m); 4.30 (2 H, s); 4.18 (1 H, d); 3.89 (1 H, bs); 3.31 (2 H, bs); 3.12 (2 H, bs); 2.96 (1 H, d); 2.65 (1 H, d); 2.55-2.40 (1 H, m); 1.83- 1.60 (8 H, m); 1.51-1.26 (4 H, m); 1.24 (3 H, s); 1.20-0.99 (2 H, m); 0.94-0.83 (6 H, m). | 664 |
| 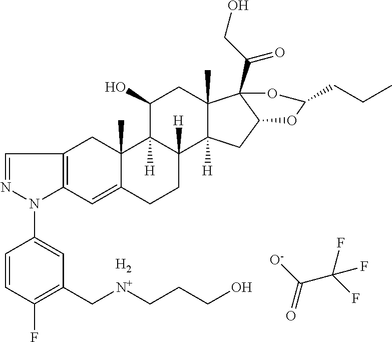 | 11 | 1- [(4aR,4bS,5S,6aS,6bS, 8R,9aR,10aS,10bS)- 1-(4-fluoro-3-{[(3- hydroxypropyl)amino] methyl}phenyl)-5- hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (400 MHz, CD₃CN) δ 7.69 (1 H, dd); 7.61-7.57 (1 H, m); 7.41 (1 H, s); 7.33 (1 H, t); 6.19 (1 H, d); 4.84 (1 H, d); 4.61 (1 H, t); 4.50 (1 H, d); 4.46-4.43 (1 H, m); 4.30 (2 H, s); 4.25 (1 H, d); 3.70 (2 H, t); 3.24 (2 H, t); 2.98 (1 H, d); 2.67 (1 H, d); 2.49 (1 H, t); 2.32 (1 H, d); 2.01 (1 H, dd); 1.80- 1.58 (6 H, m); 1.47-1.37 (2 H, m); 1.26 (3 H, s); 1.21 (1 H, dd); 1.11-0.99 (1 H, m); 0.92 (3 H, t); 0.87 (3 H, s). | 638 |
| 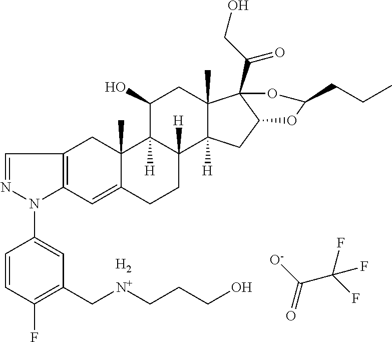 | 12 | 1- [(4aR,4bS,5S,6aS,6bS, 8S,9aR,10aS,10bS)- 1-(4-fluoro-3-{[(3- hydroxypropyl)amino] methyl}phenyl)-5- hydroxy-4a,6a-dimethyl- 8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.68 (1 H, dd); 7.61-7.55 (1 H, m); 7.39 (1 H, s); 7.31 (1 H, t); 6.18 (1 H, d); 5.23 (1 H, t); 5.14 (1 H, d); 4.58 (1 H, d); 4.44-4.40 (1 H, m); 4.29 (2 H, s); 4.18 (1 H, d); 3.68 (2 H, t); 3.22 (2 H, t); 2.96 (1 H, d); 2.65 (1 H, d); 2.55-2.40 (1 H, m); 1.91-1.63 (5 H, m); 1.51- 1.29 (4 H, m); 1.26-1.18 (4 H, m); 1.14-1.02 (1 H, m); 0.93-0.87 (6 H, m). | 638 |
| 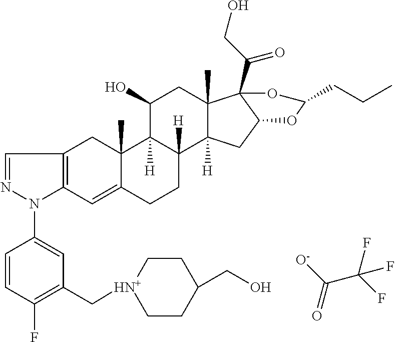 | 13 | 1- [(4aR,4bS,5S,6aS,6bS, 8R,9aR,10aS,10bS)- 1-(4-fluoro-3-{[4- (hydroxymethyl) piperidin-1- yl]methyl}phenyl)- 5-hydroxy-4a,6a- dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (400 MHz, CD₃CN) δ 7.73 (1 H, d); 7.63-7.58 (1 H, m); 7.39 (1 H, s); 7.33 (1 H, t); 6.17 (1 H, d); 4.83 (1 H, d); 4.60 (1 H, t); 4.49 (1 H, d); 4.45-4.42 (1 H, m); 4.31 (2 H, s); 4.24 (1 H, d); 3.49 (2 H, bs); 3.37- 3.33 (2 H, m); 2.99-2.85 (3 H, m); 2.66 (1 H, d); 2.53-2.43 (1 H, m); 2.33- 2.25 (1 H, m); 2.00 (1 H, dd); 1.92-1.84 (3 H, m); 1.78-1.53 (8 H, m); 1.47- 1.36 (2 H, m); 1.24 (3 H, s); 1.19 (1 H, dd); 1.08- 0.98 (1 H, m); 0.91 (3 H, t); 0.86 (3 H, s). | 678 |
| 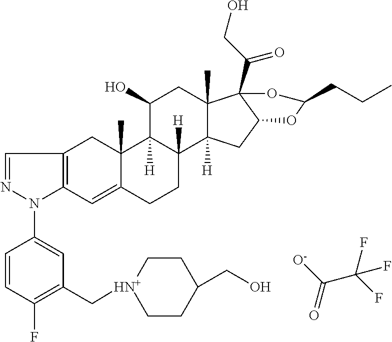 | 14 | 1- [(4aR,4bS,5S,6aS,6bS, 8S,9aR,10aS,10bS)- 1-(4-fluoro-3-{[4- (hydroxymethyl)- piperidin-1-yl]methyl} phenyl)-5-hydroxy- 4a,6a-dimethyl-8-propyl- 4,4a,4b,5,6,6a,9a,10, 10a,10b,11,12- dodecahydro[1,3]dioxolo [3′,4′]cyclopenta [1′,2′:5,6]naphtho[1,2-f] indazol-6b(1H)-yl]- 2-hydroxyethanone trifluoroacetic acid | ¹H NMR (300 MHz, CD₃CN) δ 7.74 (1 H, dd); 7.64-7.58 (1 H, m); 7.39 (1 H, s); 7.33 (1 H, t); 6.16 (1 H, d); 5.23 (1 H, t); 5.14 (1 H, d); 4.58 (1 H, d); 4.44-4.40 (1 H, m); 4.31 (2 H, s); 4.18 (1 H, d); 3.50 (2 H, bs); 3.37- 3.32 (2 H, m); 2.99-2.85 (3 H, m); 2.65 (1 H, d); 2.54-2.40 (1 H, m); 1.88- 1.43 (8 H, m); 1.40-1.28 (2 H, m); 1.26-1.17 (4 H, m); 1.15-1.02 (1 H, m); 0.93-0.87 (6 H, m). | 678 |
| 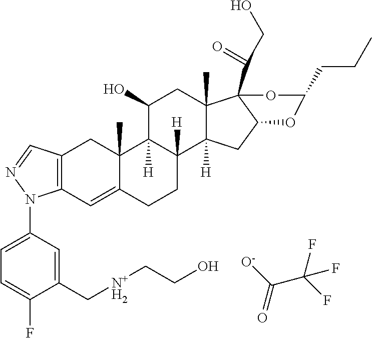 | 15 | 1- [(4aR,4bS,5S,6aS,6bS, 8R,9aR,10aS,10bS)- 1-(4-fluoro-3-{[(2- hydroxyethyl)amino]- methyl}phenyl)- 5-hydroxy-4a,6a- dimethyl-8-propyl-4,4a, 4b,5,6,6a,9a,10,- 10a,10b,11,12-dodecahydro [1,3]dioxolo- [3′,4′]cyclopenta[1′,2′:5,6] naphtho[1,2- f]indazol-6b(1H)-yl]- 2-hydroxyethanone trifluoroacetic acid | ¹H NMR (400 MHz, CD₃CN) δ 7.71-7.68 (1 H, m); 7.59-7.55 (1 H, m); 7.39 (1 H, s); 7.30 (1 H, t); 6.19 (1 H, s); 4.83 (1 H, d); 4.60 (1 H, t); 4.49 (1 H, d); 4.46-4.42 (1 H, m); 4.31 (2 H, s); 4.24 (1 H, d); 3.77 (2 H, bs); 3.28- 3.22 (1 H, m); 3.14 (2 H, bs); 2.97 (1 H, d); 2.67 (1 H, d); 2.53-2.42 (1 H, m); 1.79-1.57 (6 H, m); 1.47- 1.32 (2 H, m); 1.25 (3 H, s); 1.20 (1 H, dd); 1.09- 0.98 (1 H, m); 0.91 (3 H, t); 0.86 (3 H, s). | 624 |

### EXAMPLE 16

### 1-[(4aR,4bS,5S,6aS,6bS,8R,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone

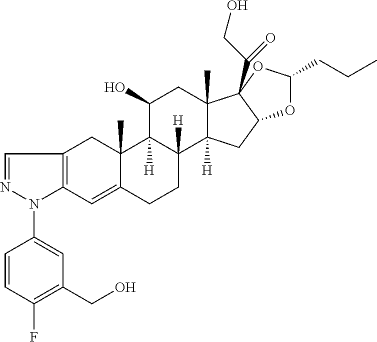

Intermediate 5 (100 mg, 0.14 mmol) was stirred in methanol with a few drops of HCl. The title compound was obtained by preparative HPLC (22 mg, 54%).

¹H NMR (400 MHz, CDCl₃) δ 7.63 (1H, dd); 7.51 (1H, s); 7.37-7.32 (1H, m); 7.16 (1H, t); 6.03 (1H, s); 4.92 (1H, d); 4.83 (2H, s); 4.59-4.50 (3H, m); 4.28 (1H, d); 3.01 (1H, d); 2.71 (1H, d); 2.50 (1H, t); 2.30 (1H, d); 2.11-1.90 (2H, m); 1.81 (1H, dd); 1.75-1.57 (5H, m); 1.48-1.39 (2H, m); 1.31 (3H, s); 1.26 (1H, dd); 1.18-1.07 (1H, m); 0.98-0.90 (6H, m). APCI-MS m/z: 581 [MH⁺].

### EXAMPLE 17

### 1-[(4aR,4bS,5S,6aS,6bS,8S,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone

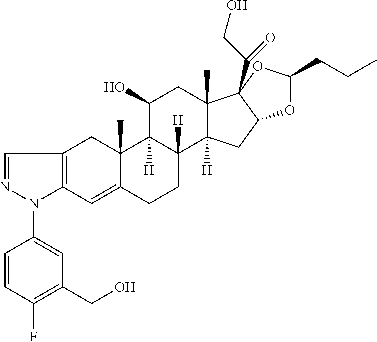

The title compound was prepared according to the procedure for Example 16.

¹H NMR (399.988 MHz, CDCl₃) δ 7.67 (1H, d); 7.59 (1H, s); 7.36-7.31 (1H, m); 7.17 (1H, t); 6.01 (1H, s); 5.23-5.18 (2H, m); 4.64 (1H, d); 4.54-4.49 (1H, m); 4.23 (1H, d); 3.03 (1H, d); 2.70 (1H, d); 2.56-2.46 (1H, m); 2.35-2.28 (1H, m); 2.10 (1H, d); 2.02-1.92 (2H, m); 1.83-1.61 (5H, m); 1.54-1.34 (4H, m); 1.32 (3H, s); 1.28-1.24 (1H, m); 1.18-1.08 (1H, m); 0.99-0.90 (6H, m). APCI-MS m/z: 581 [MH⁺].

### EXAMPLE 18

### 1-[(4aR,4bS,5S,6aS,6bS,8R,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo[3′,4′]-cyclopenta[1′,2′:5,6]naphtho[1,2-f]indazol-6b(1H)-yl]-2-hydroxyethanone

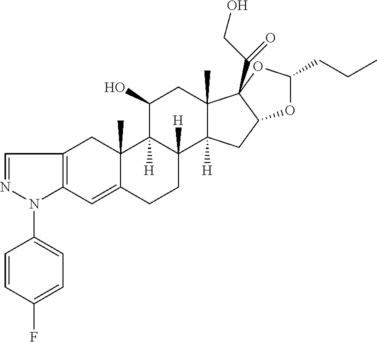

The title compound was prepared according to the same method as the preparation of intermediate 5 using 4-fluorophenylhydrazine hydrochloride. The protection group was removed by stirring in methanol with a few drops of HCl for 2 h. The two isomers were separated on a Phenomenex Gemini 5μ, C18 column (ethanol/water/1% ammonia).

¹H NMR (399.99 MHz, acetone-d₆) δ 7.59-7.55 (2H, m); 7.42 (1H, s); 7.30 (2H, t); 6.19 (1H, d); 4.86 (1H, d); 4.62 (1H, t); 4.57-4.49 (2H, m); 4.26 (1H, d); 3.04 (1H, d); 2.72 (1H, d); 2.59-2.48 (1H, m); 2.36-2.29 (1H, m); 2.03-1.71 (6H, m); 1.66-1.59 (2H, m); 1.50-1.41 (2H, m); 1.33 (3H, s); 1.25-1.21 (1H, m); 1.13-1.02 (1H, m); 0.94-0.89 (6H, m). APCI-MS m/z: 551 [MH⁺].

### EXAMPLE 19

### 1-[(4aR,4bS,5S,6aS,6bS,8S,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone

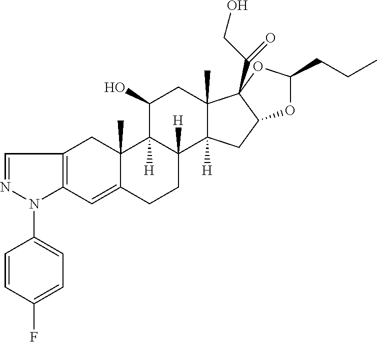

The title compound was prepared according to the procedure for Example 18.

¹H NMR (399.99 MHz, DMSO-d₆) δ 7.53-7.49 (2H, m); 7.45 (1H, s); 7.37-7.32 (2H, m); 6.10 (1H, s); 5.21 (1H, t); 5.07 (1H, d); 5.02 (1H, t); 4.48 (1H, dd); 4.40 (1H, d); 4.33-4.30 (1H, m); 4.08 (1H, dd); 2.93 (1H, d); 2.61 (1H, d); 2.31-2.24 (1H, m); 1.87-1.53 (6H, m); 1.45-1.36 (2H, m); 1.32-1.23 (2H, m); 1.21 (3H, s); 1.15-1.10 (1H, m); 1.07-0.98 (1H, m); 0.89-0.82 (6H, m). APCI-MS m/z: 551 [MH⁺].

### Intermediate 7

### (3Z,4aR,4bS,5S,6aS,6bS,9aR,10aS,10bS)-6b-({[tert-Butyl(dimethyl)silyl]oxy}acetyl)-5-hydroxy-3-(hydroxymethylene)-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-2-one

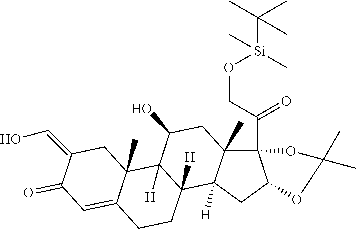

Intermediate 7 was prepared according to the procedure for intermediate 3 starting from (4aR,4bS,5S,6aS,6bS,9aR,10aS,10bS)-6b-glycoloyl-5-hydroxy-4a,6a,8,8-tetramethyl-4a,4b,5,6,6a,6b,9a,10,10a,10b,11,12-dodecahydro-2H-naphtho[2′,1′:4,5]indeno[1,2-d][1,3]dioxol-2-one (desonide). APCI-MS m/z: 561 [MH⁺].

### Intermediate 8

### 2-{[tert-Butyl(dimethyl)silyl]oxy}-1-[(4aR,4bS,5S,6aS,6bS,9aR,10aS,10bS)-1-(4-fluorophenyl)-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]ethanone

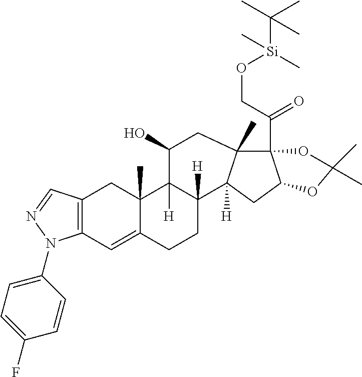

Intermediate 8 was prepared according to the procedure for the preparation of intermediate 5 using 4-fluorophenylhydrazine hydrochloride. APCI-MS m/z: 651 [MH⁺].

### EXAMPLE 20

### 1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone

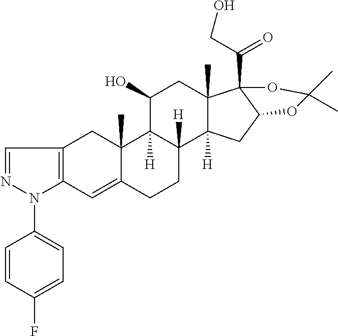

Intermediate 8 (23 mg, 0.035 mmol) was dissolved in THF (2 ml), Acetic acid (10 μl) was added followed by tertrabutylammonium fluoride (32 mg, 0.11 mmol). The mixture was stirred overnight. The crude product was purified by preparative HPLC using MeCN/water. Pure fractions were evaporated to give the title compound as solid material (15 mg, 80%).

¹H NMR (299.949 MHz, CDCl₃) δ 7.44 (2H, tt); 7.40 (1H, s); 7.18-7.12 (2H, m); 6.05 (1H, d); 5.06 (1H, d); 4.67 (1H, dd); 4.50 (1H, s); 4.18 (1H, dd); 3.09 (1H, t); 2.96 (1H, d); 2.68 (1H, d); 2.55-2.41 (1H, m); 2.31-2.18 (1H, m); 2.06-1.87 (2H, m); 1.79-1.58 (4H, m); 1.45 (3H, s); 1.32-1.04 (8H, m); 0.88 (3H, d). APCI-MS m/z: 537 [MH⁺].

### EXAMPLE 21

### 1-[(4aR,4bS,5S,6aS,6bS,9aR,10aS,10bS)-1-[4-Fluoro-3-(hydroxymethyl)phenyl]-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone

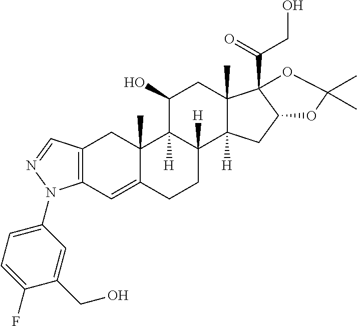

Intermediate 7 (0.449 g, 0.8 mmol) and potassium acetate (0.157 g, 1.60 mmol) were mixed in ethanol (35 ml). (2-Fluoro-5-hydrazinylphenyl)methanol hydrochloride (0.200 g, 1.04 mmol) was added and the reaction mixture was heated under microwave irradiation at 76° C. for 60 minutes. Formic acid (6.14 ml, 80.0 mmol) was added to the reaction mixture at room temperature. After 10 minutes the solvents were evaporated and the crude product was purified on a “Kromasil” (trade mark) column and was eluted with acetonitrile/water/0.1% TFA. Pure fractions were evaporated to give 95 mg of the title compound as solid material (95 mg, 21%).

¹H NMR (400 MHz, CDCl₃) δ 7.65 (1H, dd); 7.54 (1H, s); 7.36-7.31 (1H, m); 7.16 (1H, t); 6.02 (1H, s); 5.07 (1H, d); 4.83 (2H, s); 4.69 (1H, d); 4.55-4.52 (1H, m); 4.20 (1H, d); 3.02 (1H, d); 2.72 (1H, d); 2.55-2.46 (1H, m); 2.10-1.92 (4H, m); 1.79 (1H, d); 1.70-1.60 (3H, m); 1.46 (3H, s); 1.32 (3H, s); 1.25 (1H, dd); 1.16 (3H, s); 0.88 (3H, s). APCI-MS m/z: 567 [MH⁺].

### Intermediate 9

### 1-[(4aR,4bS,5S,6aS,6bS,9aR,10aS,10bS)-1-(3-Bromo-4-fluorophenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-{[tert-butyl(dimethyl)silyl]oxy}ethanone

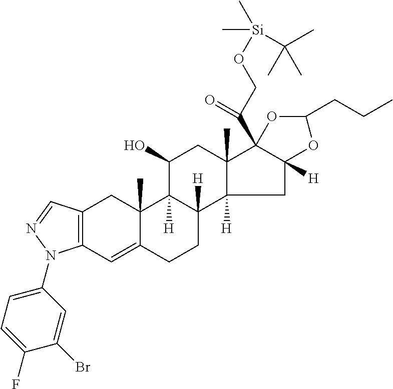

This intermediate was prepared from intermediate 3 and (3-bromo-4-fluorophenyl)hydrazine hydrochloride according to the same procedure as the preparation of intermediate 5. In order to detect the product on LC-MS the protection group was removed in the LC-MS sample with methanol/10% HCl. APCI-MS m/z: 629, 631 [MH⁺].

### Intermediate 10

### Methyl 5-[(4aR,4bS,5S,6aS,6bS,9aR,10aS,10bS)-6b-({[tert-butyl(dimethyl)silyl]oxy}acetyl)-5-hydroxy-4a,6a-dimethyl-8-propyl-4a,4b,5,6,6a,6b,9a,10,10a,10b,11,12-dodecahydro[1,3]dioxolo-[3′,4′]cyclopenta[1′,2′:5,6]naphtho[1,2-f]indazol-1(4H)-yl]-2-fluorobenzoate

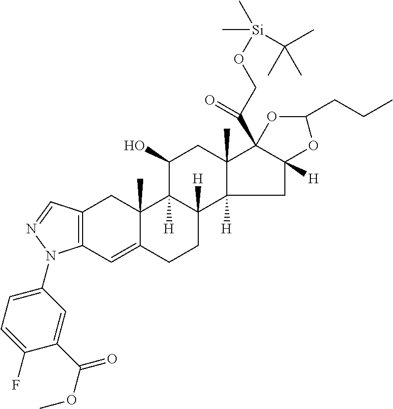

Intermediate 9 (260 mg, 0.35 mmol) was dissolved in DMF (2 ml) and methanol (2 ml). Palladium (II) acetate (11 mg, 0.05 mmol), 1,3-bis(diphenylphosphino)propane (33 mg, 0.08 mmol) and DIEA (174 μl, 1.05 mmol) were added. The mixture was heated in an autoclave at 110° C. under carbon monoxide pressure (5 bar) for 40 h. The mixture was partitioned between water and EtOAc. The organic phase was evaporated and the crude product was purified on silica (EtOAc/heptane 1:3) to give the title compound (217 mg, 86%). In order to detect the product on LC-MS the protection group was removed in the LC-MS sample with methanol/10% HCl. APCI-MS m/z: 609 [MH⁺].

### EXAMPLE 22

### Methyl 2-fluoro-5-[(4aR,4bS,5S,6aS,6bS,8R,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]benzoate

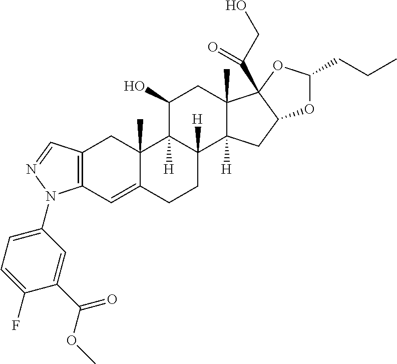

A solution of intermediate 10 (155 mg, 0.21 mmol) in ethanol (3 ml) was treated with 3N HCl (0.5 ml). The suspension was stirred at room temperature for 0.5 h. The crude product was purified by preparative HPLC (ethanol/water/0.1% TFA) to give the title compound (3 mg, 5%).

¹H NMR (399.988 MHz, CDCl₃) δ 8.04 (1H, dd); 7.69-7.64 (1H, m); 7.42 (1H, s); 7.25 (1H, t); 6.07 (1H, d); 4.92 (1H, d); 4.58-4.49 (3H, m); 4.27 (1H, dd); 3.95 (3H, s); 3.04-2.95 (2H, m); 2.70 (1H, d); 2.54-2.44 (1H, m); 2.33-2.26 (1H, m); 2.09-1.89 (2H, m); 1.81 (1H, dd); 1.75-1.56 (6H, m); 1.48-1.38 (2H, m); 1.30 (3H, s); 1.27-1.20 (1H, m); 1.18-1.06 (1H, m); 0.96-0.89 (6H, m). APCI-MS m/z: 609 [MH⁺].

### EXAMPLE 23

### Methyl 2-fluoro-5-[(4aR,4bS,5S,6aS,6bS,8S,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]benzoate

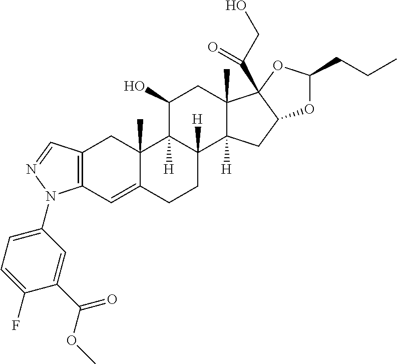

The title compound was prepared according to the procedure for Example 22.

¹H NMR (399.988 MHz, cdcl3) δ 8.04 (1H, dd); 7.69-7.64 (1H, m); 7.42 (1H, s); 7.25 (1H, t); 6.07 (1H, d); 5.24-5.18 (2H, m); 4.64 (1H, d); 4.51 (1H, s); 4.23 (1H, d); 3.95 (3H, s); 3.05-2.95 (2H, m); 2.68 (1H, d); 2.54-2.44 (1H, m); 2.33-2.26 (1H, m); 2.09 (1H, dd); 2.02-1.90 (2H, m); 1.86-1.44 (6H, m); 1.42-1.33 (2H, m); 1.30 (3H, s); 1.28-1.23 (1H, m); 1.19-1.07 (1H, m); 0.98 (3H, s); 0.92 (3H, t).

APCI-MS m/z: 609 [MH⁺].

### Intermediate 11

### (4aR,4bS,5S,6aS,6bS,9aR,10aS,10bS,12S)-12-Fluoro-6b-glycoloyl-5-hydroxy-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-2-one

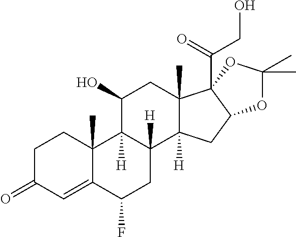

Intermediate 11 was prepared from (4aR,4bS,5S,6aS,6bS,9aR,10aS,10bS,12S)-12-fluoro-6b-glycoloyl-5-hydroxy-4a,6a,8,8-tetramethyl-4a,4b,5,6,6a,6b,9a,10,10a,10b,11,12-dodecahydro-2H-naphtho[2′,1′:4,5]indeno[1,2-d][1,3]dioxol-2-one (flunisolide) according to the same procedure as the preparation of intermediate 1.

APCI-MS m/z: 437 [MH⁺].

### Intermediate 12

### (4aR,4bS,5S,6aS,6bS,9aR,10aS,10bS,12S)-6b-({[tert-Butyl(dimethyl)silyl]oxy}acetyl)-12-fluoro-5-hydroxy-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-2-one

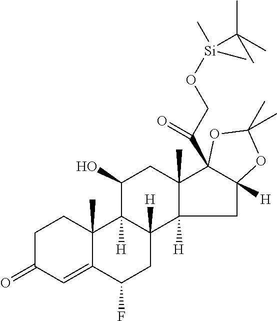

Intermediate 12 was prepared from intermediate 11 according to the procedure for intermediate 2. APCI-MS m/z: 551 [MH⁺].

### Intermediate 13

### (4aR,4bS,5S,6aS,6bS,10aS,10bS)-6b-({[tert-Butyl(dimethyl)silyl]oxy}acetyl)-12-fluoro-5-hydroxy-4a,6a,8,8-tetramethyl-2-oxo-3,4,4a,4b,5,6,6a,6b,9a,10,10a,10b,11,12-tetradecahydro-2H-naphtho[2′,1′:4,5]indeno[1,2-d][1,3]dioxole-3-carbaldehyde

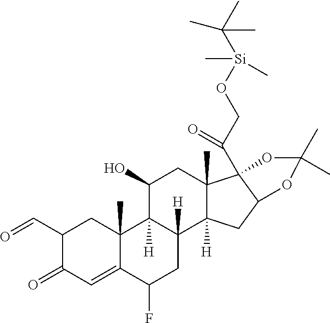

In a 10 ml vessel intermediate 12 (100 mg, 0.18 mmol) and ethyl formate (30 μl, 0.36 mmol) were mixed in toluene and purged with argon. Sodium hydride (79 mg, 1.8 mmol, 55-60% in oil) was added and the mixture was stirred for 1 h. The reaction mixture was then purified on a silica column with the product eluting with CH₂Cl₂. The solution was concentrated and used as such in the next step. APCI-MS m/z: 579 [MH⁺].

### Intermediate 14

### 2-{[tert-Butyl(dimethyl)silyl]oxy}-1-[(4aR,4bS,5S,6aS,6bS,10aS,10bS)-1-(4-fluorophenyl)-5,12-dihydroxy-4a,6a,8,8-tetramethyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]ethanone

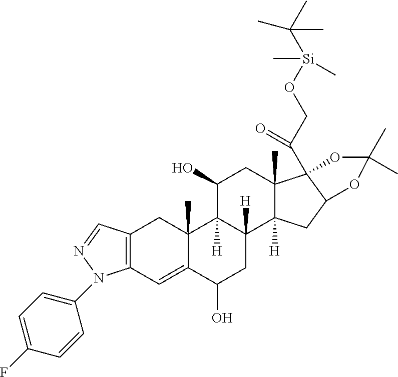

In a 10 ml round-bottomed flask, intermediate 13 (80 mg, 0.14 mmol), 4-fluorophenylhydrazine hydrochloride (22.47 mg, 0.14 mmol) and potassium acetate (8.6 μl, 0.14 mmol) were dissolved in a mixture of acetic acid (1 ml), water (1 ml) and ethanol (1 ml) to give a brown solution. The mixture was stirred for 1 hour at room temperature. The crude product was purified by preparative HPLC using MeCN/water to give 10 mg of the desired product. APCI-MS m/z: 667 [MH⁺].

### EXAMPLE 24

### 1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b-decahydro[1,3]dioxolo[3′,4′]cyclopenta-[1′,2′:5,6]naphtho[1,2-f]indazol-6b(1H)-yl]-2-hydroxyethanone

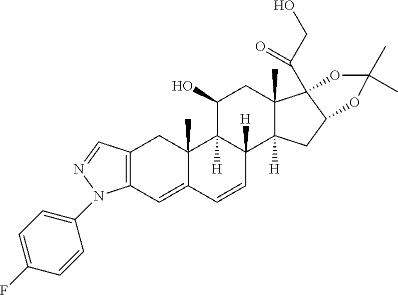

A solution of intermediate 14 (10 mg, 0.015 mmol) in ethanol (1 ml) was treated with HCl (0.15 mmol). The suspension was stirred at room temperature for 0.5 h. The crude material was purified by preparative HPLC to obtain the title compound (2 mg, 25%).

¹H NMR (400 MHz, CDCl₃) δ 7.50-7.44 (3H, m); 7.19-7.14 (2H, m); 6.12-6.08 (2H, m); 5.81 (1H, d); 5.11 (1H, d); 4.69 (1H, dd); 4.52 (1H, s); 4.20 (1H, dd); 3.04-2.94 (2H, m); 2.76 (1H, t); 2.70-2.65 (1H, m); 2.07 (1H, dd); 1.95 (1H, dd); 1.87-1.64 (3H, m); 1.51-1.42 (4H, m); 1.23-1.19 (4H, m); 1.16 (3H, s); 0.92 (3H, s). APCI-MS m/z: 535 [MH⁺].

### Intermediate 15

### (4aR,4bS,5S,6aS,6bS,9aR,10aS,10bS)-6b-carboxy-5-hydroxy-4a,6a-dimethyl-8-propyl-3,4,4a,4b,5,6,6a,6b,9a,10,10a,10b,11,12-tetradecahydro-2H-naphtho[2′,1′:4,5]indeno[1,2-d][1,3]dioxol-2-one

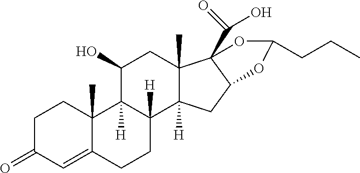

Periodic acid (5.4 g, 23.69 mmol) dissolved in water (20 ml) was added to a solution of intermediate 1 (9.1 g, 21.04 mmol) in dioxane (60 ml) and the reaction mixture was stirred at r.t. in an open flask for 4.5 h. After reaction completion, the solution was poured carefully into cold saturated aqueous sodium bicarbonate and evaporated. The residue was dissolved in 250 ml methylene chloride and washed with aqueous 1M NaOH. The aqueous phase was acidified with conc. HCl and extracted with 2*250 ml EtOAc, dried and the solvent was subsequently evaporated. The residue was dissolved in a minimum amount of EtOAc and precipitated by addition of petroleum ether (40-60) to give 4.5 g (10.75 mmol, 51%) of the desired product. APCI-MS m/z: 419 [MH⁺]

### Intermediate 16

### (4aR,4bS,5S,6aS,6bS,9aR,10aS,10bS)-6b-carboxy-5-hydroxy-4a,6a-dimethyl-2-oxo-8-propyl-3,4,4a,4b,5,6,6a,6b,9a,10,10a,10b,11,12-tetradecahydro-2H-naphtho[2′,1′:4,5]indeno[1,2-d][1,3]dioxole-3-carbaldehyde

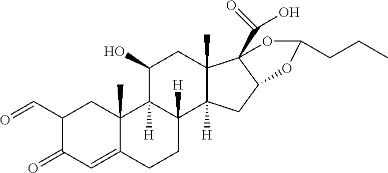

Prepared according to intermediate 3 using intermediate 15. APCI-MS m/z: 447 [MH⁺].

### Intermediate 17

### (4aR,4bS,5S,6aS,6bS,9aR,10aS,10bS)-6b-carboxy-1-(4-fluorophenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol

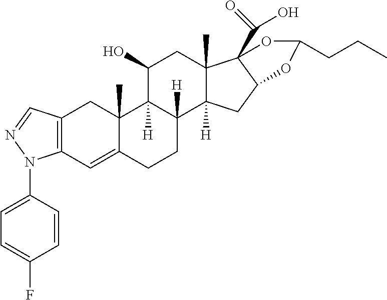

Prepared according to intermediate 5 using intermediate 16. APCI-MS m/z: 537 [MH⁺].

### Intermediate 18

### (4aR,46S,5S,6aS,6bS,9aR,10aS,10bS)-1-(4-fluorophenyl)-5-hydroxy-4a,6a-dimethyl-6b-(N,N-dimethylaminocarbonylthiocarbonyl)-8-propyl-4,4a,4b,5,6,6a,9a,10,10a,10b,11,12-dodecahydro[1,3]dioxolo[3′,4′]-cyclopenta[1′,2′:5,6]naphtho[1,2-f]indazol

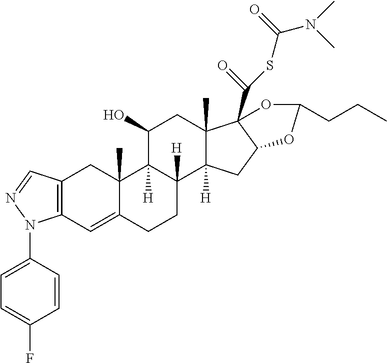

A solution of intermediate 17 (150 mg, 0.28 mmol) and N,N-dimethylthiocarbamoyl chloride (70.0 mg, 0.57 mmol) in acetone was treated with triethylamine (90 μl, 0.64 mmol), sodium iodide (3.81 μl, 0.09 mmol) and water (20 μl). The mixture was stirred for 4 hrs at r.t. The solvent was removed in vacuo and the resulting residue taken up in EtOAc and washed with aqueous sodium hydrogen carbonate. The organic phase was dried with sodium hydrogen sulphate, filtered and concentrated to afford 126 mg (0.20 mmol; 72%) of a colourless solid which was used without further purification. APCI-MS m/z: 624 [MH⁺]

### Intermediate 19

### (4aR,4bS,5S,6aS,6bS,9aR,10aS,10bS)-1-(4-fluorophenyl)-5-hydroxy-6b-(mercaptocarbonyl)-4a,6a-dimethyl-8-propyl-4,4a,4b,5,6,6a,9a,10,10a,10b,11,12-dodecahydro[1,3]-dioxolo[3′,4′]cyclopenta[1′,2′:5,6]naphtho[1,2-f]indazol

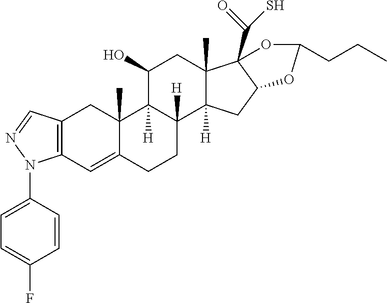

A solution of intermediate 18 (119 mg, 0.19 mmol) in N,N-dimethylacetamide (4 ml) was treated with NaSH monohydrate (105 mg, 0.19 mmol). The solution was stirred at rt for 4 hrs. The mixture was poured onto cold 1M aq. HCl. The resulting yellowish precipitate (85 mg, 0.15 mmol, 76%) was filtered and used without further purification. APCI-MS m/z: 553 [MH⁺].

### EXAMPLE 25

### (4aR,4bS,5S,6aS,6bS,8R,9aR,10aS,10bS)-6b-(cyanomethylthiocarbonyl)-1-(4-fluorophenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol

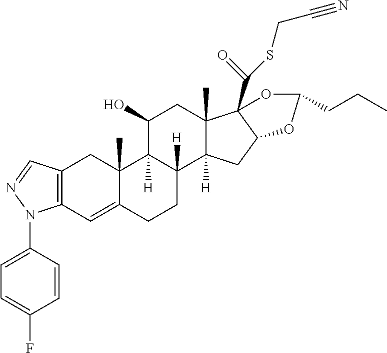

A mixture of intermediate 19 (85 mg, 15 mmol), bromoacetonitrile (20.29 mg, 0.17 mmol) and anhydrous potassium carbonate (25.5 mg, 0.18 mmol) in acetone (4 mL) was stirred at room temperature for 3 hrs. On completion the solvent was removed and the resulting residue was taken up in dichloromethane and washed with water. The organic phase was dried in Na₂SO₄, filtered and concentrated. The resulting residue was purified by preparative HPLC to give the (R)-epimer of the desired product 18 mg (0.03 mmol, 20%).

¹H NMR (400 MHz, cd3cn) δ 7.51-7.48 (2H, m); 7.39 (1H, s); 7.27-7.22 (2H, m); 6.13 (1H, d); 4.78 (1H, d); 4.72 (1H, t); 4.46 (1H, d); 3.70 (2H, d); 3.17-3.10 (3H, m); 2.96 (1H, d); 2.66 (1H, d); 2.48 (1H, t); 2.00 (1H, dd); 1.90-1.64 (6H, m); 1.50-1.42 (2H, m); 1.24 (3H, s); 1.19 (1H, dd); 1.09-0.98 (1H, m); 0.97 (3H, s); 0.94 (3H, t). APCI-MS m/z: 592 [MH⁺].

### Intermediate 20

### (8S,9S,10R,11S,13S,14S,16R,17S)-11,16,17-Trihydroxy-17-(2-hydroxyacetyl)-10,13-dimethyl-2,6,7,8,9,11,12,14,15,16-decahydro-1H-cyclopenta[a]phenanthren-3-one

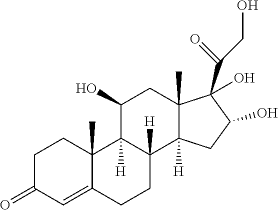

A mixture of Chlorotris(triphenylphosphine)rhodium(I) (7 g, 7.56 mmol) in ethanol (600 ml) and toluene (250 ml) was degassed with nitrogen three times. (8S,9S,10R,11S,13S,14S,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (10 g, 26.56 mmol) was added and the mixture hydrogenated at atmospheric pressure and r.t. for 72 hrs. The solvents were removed in vacuo and the residue was taken up in CH₂Cl₂. The undissolved material was filtered off, washed with CH₂Cl₂ and the solvent removed under reduced pressure to give 8.5 g (22.46 mmol, 85%) of the desired product as a light yellow solid. APCI-MS m/z: 418 [MH⁺].

### Intermediate 21

### (4aR,4bS,5S,6aS,6bS,9aR,10aS,10bS)-6b-Glycoloyl-5-hydroxy-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-2-one

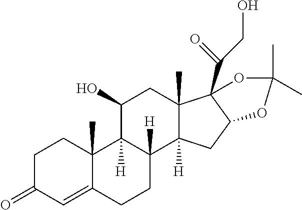

To a mixture of intermediate 20 (8.5 g, 22.46 mmol) in acetone (50 ml) was added perchloric acid (10.35 μl, 0.17 mmol) and stirred until reaction completion. The yellowish clear solution was poured into pre-cooled saturated aq. NaHCO₃ and the resulting precipitate was filtered and dried to give 8.5 g of the desired product (20.31 mmol, 90%) as an off-white solid. APCI-MS m/z: 419 [MH⁺].

### Intermediate 22

### (4aR,4bS,5S,6aS,6bS,9aR,10aS,10bS)-6b-carboxy-5-hydroxy-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-2-one

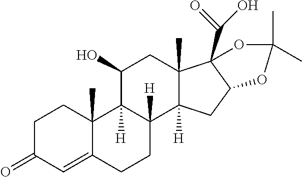

Prepared according to intermediate 15 using intermediate 21. APCI-MS m/z: 405 [MH⁺].

### Intermediate 23

### (4aR,4bS,5S,6aS,6bS,9aR,10aS,10bS)-6b-carboxy-5-hydroxy-2-oxo-4a,6a,8,8-tetramethyl-3,4,4a,4b,5,6,6a,6b,9a,10,10a,10b,11,12-tetradecahydro-2H-naphtho[2′,1′:4,5]indeno[1,2-d][1,3]dioxol-3-carbaldehyde

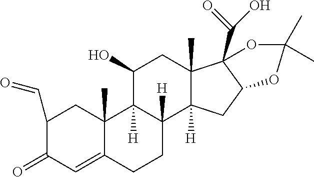

Prepared according to intermediate 3 using intermediate 22. APCI-MS m/z: 433 [MH⁺].

### Intermediate 24

### (4aR,4bS,5S,6aS,6bS,9aR,10aS,10bS)-6b-carboxy-1-(4-fluorophenyl)-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol

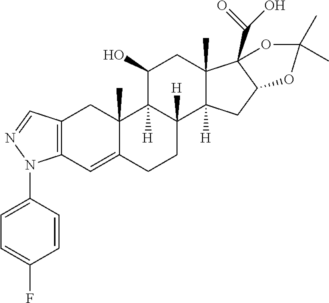

Prepared according to intermediate 5 using intermediate 23. APCI-MS m/z: 523 [MH⁺].

### Intermediate 25

### (4aR,4bS,5S,6aS,6bS,9aR,10aS,10bS)-1-(4-fluorophenyl)-5-hydroxy-4a,6a,8,8-tetramethyl-6b-(N,N-dimethylcarbonylthiocarbonyl)-4,4a,4b,5,6,6a,9a,10,10a,10b,11,12-dodecahydro[1,3]dioxolo[3′,4′]-cyclopenta[1′,2′:5,6]naphtho[1,2-f]indazol

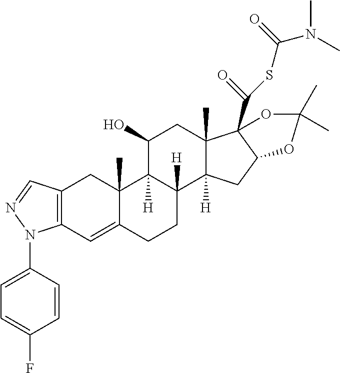

Prepared according to intermediate 18 using intermediate 24. APCI-MS m/z: 610 [MH⁺].

### Intermediate 26

### (4aR,4bS,5S,6aS,6bS,9aR,10aS,10bS)-1-(4-fluorophenyl)-5-hydroxy-6b-(mercaptocarbonyl)-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol

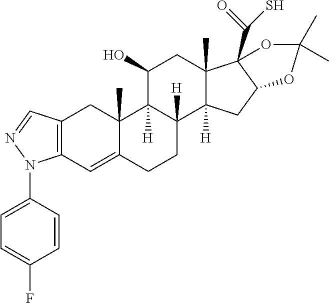

A suspension of intermediate 25 (250 mg, 0.41 mmol) and potassium carbonate (113 mg, 0.82 mmol) in methanol (5 ml) was stirred at room temperature. On reaction completion water was added and the solution was washed with toluene. The aqueous phase was acidified with 2N HCl to about pH 1.0 and the resulting precipitate was filtered, washed with water and dried in air to give 125 mg (0.23 mmol, 57%) of the title compound. APCI-MS m/z: 539 [MH⁺]

### EXAMPLE 26

### (4aR,4bS,5S,6aS,6bS,9aR,10aS,10bS)-6b-(cyanomethylcarbonyl)-1-(4-fluorophenyl)-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol

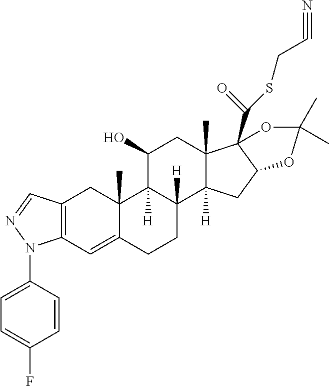

Prepared according to Example 25 using intermediate 26. APCI-MS m/z: 578 [MH⁺].

### EXAMPLE 27

### (4aR,4bS,5S,6aS,6bS,8R,9aR,10aS,10bS)-8-cyclohexyl-6b-(cyanomethylcarbonyl)-1-(4-fluorophenyl)-5-hydroxy-4a,6a-dimethyl-4,4a,4b,5,6,6a,9a,10,10a,10b,11,12-dodecahydro[1,3]dioxolo[3′,4′]cyclopenta[1′,2′:5,6]naphtho[1,2-f]indazol

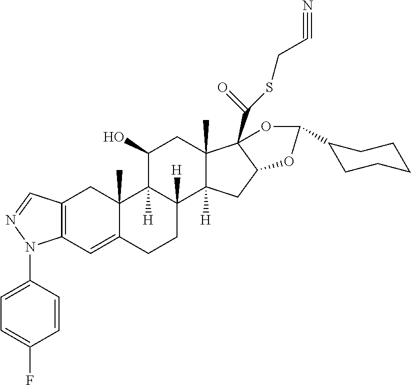

To 21 mg (0.036 mmol) of the compound of Example 26 was added 1-butyl-3-methylimidazolium hexafluorophosphate (30.0 μl, 0.15 mmol) followed by cyclohexanecarboxaldehyde (6.0 μl, 0.05 mmol) and perchloric acid, 70% (9.0 μl, 0.15 mmol) diluted in dichloromethane (1 ml). The reaction was stirred at 28° C. for 10 min, diluted with 5 ml dichloromethane and then poured into 5 ml sodium bicarbonate solution and washed with water. The organic phase was dried over sodium sulphate, filtered and concentrated under reduced pressure. Purification on HPLC gave 4 mg (0.006 mmol, 17%) of the desired product.

¹H NMR (400 MHz, cd3cn) δ 7.76 (1H, s); 7.69-7.66 (2H, m); 7.21-7.17 (2H, m); 6.14 (1H, d); 4.79 (1H, d); 4.48-4.45 (2H, m); 3.70 (2H, d); 3.02 (1H, d) 2.67 (2H, d); 2.57-2.48 (1H, m); 2.37-2.32 (1H, m); 2.07-1.98 (1H, m); 1.81-1.60 (10H, m); 1.26 (3H, s); 1.24-1.0 (7H, m); 0.98 (3H, t). APCI-MS m/z: 632 [MH⁺].

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10× (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10× (Panvera, Part number P2814, stored at −70° C. initially but once thawed stored at room temperature). Avoid repeated freeze/thaws for all reagents. The GR Screening buffer 10× comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). 0% control was 100% DMSO and 100% control was 10 μM Dexamethasone. Background solution (8 μL; assay buffer 10×, Stabilizing Peptide, DTT and ice cold MQ water) was added to the background wells. GS Red solution (7 μL; assay buffer 10×, Stabilizing Peptide, DTT, GS Red and ice cold water) was added to all wells except background wells. GR solution (7 μL; assay buffer 10×, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nm and a dichroic mirror at 561 nm). The IC₅₀ values were calculated using XLfit model 205 and are shown in Table 1.

TABLE 1

| | Inhibition of | | Inhibition of |
|---|---|---|---|
| Example | GR binding, | Example | GR binding, |
| No. | IC₅₀ (nM) | No. | IC₅₀ (nM) |
| 1 | 8 | 2 | 8.7 |
| 3 | 37 | 4 | 21 |
| 5 | 77 | 6 | 72 |
| 7 | 12 | 8 | 16 |
| 9 | 21 | 10 | 67 |
| 11 | 34 | 12 | 76 |
| 13 | 32 | 14 | 47 |
| 15 | 42 | 16 | 3.1 |
| 17 | 6.6 | 18 | 2.3 |
| 19 | 2.7 | 20 | 1.3 |
| 21 | 5.5 | 22 | 5.9 |
| 23 | 5.9 | 24 | 3.8 |
| 25 | 12 | | |

1. A compound of formula 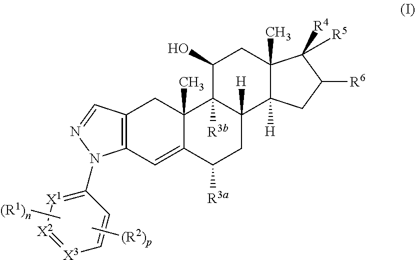
wherein
X¹, X² and X³ each represent CH or, alternatively, one of X¹, X² and X³ may additionally represent a nitrogen atom;
n and p each independently represent 0 or 1;
R¹ represents a halogen atom or a methyl or a methoxy group;
R² represents —CO₂CH₃, a halogen atom, or a methyl group optionally substituted by a hydroxyl or a —NR⁷R⁸ group;
R^{3a} represents a hydrogen atom and R^{3b} represents a hydrogen or fluorine atom;
R⁴ represents —C(O)CH₂OH or —C(O)—Y—CH₂R⁹;
R⁵ and R⁶ together with the carbon atoms to which they are attached form a 1,3-dioxolanyl group which is optionally substituted by at least one substituent selected from C₁-C₃ alkyl and C₃-C₈ cycloalkyl;
R⁷ and R⁸ each independently represent a hydrogen atom, or a C₁-C₃ alkyl or a C₁-C₃ hydroxyalkyl group, or
R⁷ and R⁸ together with the nitrogen atom to which they are attached form a 3- to 8-membered saturated or partially saturated heterocyclic ring optionally containing a further ring heterogroup selected from nitrogen, S(O)ₘ and oxygen, the heterocyclic ring being optionally substituted by at least one substituent selected from hydroxyl, C₁-C₃ alkyl and C₁-C₃ hydroxyalkyl;
m is 0, 1 or 2;
Y represents an oxygen or sulphur atom or a group >NH; and
R⁹ represents a hydrogen or a halogen atom or a methyl or a cyano group; or
a pharmaceutically acceptable salt thereof.

2. A compound according to claim 1, wherein X¹, X² and X³ each represent CH.

3. A compound according to claim 1, wherein n is 1 and R¹ represents a halogen atom.

4. A compound according to claim 1, wherein p is 1 and R² represents a methyl group optionally substituted by a hydroxyl or a —NR⁷R⁸ group.

5. A compound according to claim 1, wherein R⁴ represents —C(O)CH₂OH.

6. A compound according to claim 1, wherein R⁴ represents —C(O)—S—CH₂CN.

7. A compound according to claim 1, wherein R⁵ and R⁶ together with the carbon atoms to which they are attached form a 1,3-dioxolanyl group which is substituted by one or two C₁-C₃ alkyl groups.

8. A compound according to claim 1, wherein R⁷ and R⁸ each independently represent a hydrogen atom, or a C₁-C₃ alkyl or a C₁-C₃ hydroxyalkyl group.

9. A compound according to claim 1, wherein R⁷ and R⁸ together with the nitrogen atom to which they are attached form a 5- to 6-membered saturated heterocyclic ring optionally containing a further ring heterogroup selected from nitrogen, S(O)ₘ and oxygen, the heterocyclic ring being optionally substituted by at least one substituent selected from hydroxyl, methyl and hydroxymethyl.

10. A compound according to claim 1 being:
1-[(4aR,4bS,5S,6aS,6bS,8R,9aR,10aS,10bS)-1-[4-fluoro-3-(morpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-(4aR,4bS,5S,6aS,6bS,8S,9aR,10aS,10bS)-1-[4-fluoro-3-(morpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-fluoro-3-(pyrrolidin-1-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{4-fluoro-3-[(4-methylpiperazin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4]cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-{4-fluoro-3-[(4-methylpiperazin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{3-[(diethylamino)methyl]-4-fluorophenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-fluoro-3-(thiomorpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-[4-fluoro-3-(thiomorpholin-4-ylmethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-{4-fluoro-3-[(4-hydroxypiperidin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-{4-fluoro-3-[(4-hydroxypiperidin-1-yl)methyl]phenyl}-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[(3-hydroxypropyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-fluoro-3-{[(3-hydroxypropyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[4-(hydroxymethyl)piperidin-1-yl]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-fluoro-3-{[4-(hydroxymethyl)piperidin-1-yl]methyl}phenyl)-5-hydroxy-4a,6a-dimethyl-8-propyl-4,4a,4b,5,6,6a,9a,10,10a,10b,11,12-dodecahydro[1,3]dioxolo[3′,4]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-fluoro-3-{[(2-hydroxyethyl)amino]methyl}phenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta-[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-[4-Fluoro-3-(hydroxymethyl)phenyl]-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
1-[(4aR,4bS,5S,6aS,6bS,8R,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo[3′,4′]-cyclopenta[1′,2′:5,6]naphtho[1,2-f]indazol-6b(1H)-yl]-2-hydroxyethanone;
1-[(4aR,4bS,5S,6aS,6bS,8S,9aR,10aS,10bS)-1-(4-Fluorophenyl)-5-hydroxy-4a,6a-dimethyl-8-propyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone;
1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b,11,12-dodecahydro[1,3]dioxolo-[3′,4′]cyclopenta[1′,2′:5,6]naphtho[1,2-f]indazol-6b(1H)-yl]-2-hydroxyethanone;
1-[(4aR,4bS,5S,6aS,6bS,9aR,10aS,10bS)-1-[4-Fluoro-3-(hydroxymethyl)phenyl]-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol-6b(1H)-yl]-2-hydroxyethanone;
Methyl 2-fluoro-5-[(4aR,4bS,5S,6aS,6bS,8R,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,′:5,6]naphtho[1,2-f]indazol-1(4H)-yl]benzoate;
Methyl 2-fluoro-5-[(4aR,4bS,5S,6aS,6bS,8S,9aR,10aS,10bS)-6b-glycoloyl-5-hydroxy-4a,6a-dimethyl-8-propyl-4a,4b,5,6,6a,6b,9a,10,10a,10b,11,12-dodecahydro[1,3]dioxolo[3′,4′]cyclopenta[1′,2′:5,6]naphtho[1,2-f]indazol-1(4H)-yl]benzoate;
1-[(4aR,4bS,5S,6aS,6bS,9aR,10aS,10bS)-1-(4-Fluorophenyl)-5-hydroxy-4a,6a,8,8-tetramethyl-4,4a,4b,5,6,6a,9a,10,10a,10b-decahydro[1,3]dioxolo[3′,4′]cyclopenta-[1′,2′:5,6]naphtho[1,2-f]indazol-6b(1H)-yl]-2-hydroxyethanone;
(4aR,4bS,5S,6aS,6bS,8R,9aR,10aS,10bS)-6b-(cyanomethylthiocarbonyl)-1-(4-fluorophenyl)-5-hydroxy-4a,6a-dimethyl-8-propyl-4,4a,4b,5,6,6a,9a,10,10a,10b,11,12-dodecahydro[1,3]dioxolo[3′,4′]cyclopenta[1′,2′:5,6]naphtho[1,2-f]indazol;
(4aR,4bS,5S,6aS,6bS,9aR,10aS,10bS)-6b-(cyanomethylthiocarbonyl)-1-(4-fluorophenyl)-5-hydroxy-4a,6a,8,8-tetramethyl-4,4a,4b,5,6,6a,9a,10,10a,10b,11,12-dodecahydro[1,3]dioxolo[3′,4′]cyclopenta[1′,2′:5,6]naphtho[1,2-f]indazol; or
(4aR,4bS,5S,6aS,6bS,8R,9aR,10aS,10bS)-8-cyclohexyl-6b-(cyanomethylthiocarbonyl)-1-(4-fluorophenyl)-5-hydroxy-4a,6a-dimethyl-4,4a,4b,5,6,6a,9a,10,10a,10b,11,12-dodecahydro[1,3]dioxolo[3′,4′]cyclopenta[1′,2′:5,6]naphtho[1,2-f]indazol;
or a pharmaceutically acceptable salt thereof.

11. A process for the preparation of a compound of formula (I) or a pharmaceutically acceptable salt thereof as defined in claim 1 which comprises reacting a compound of formula (II) 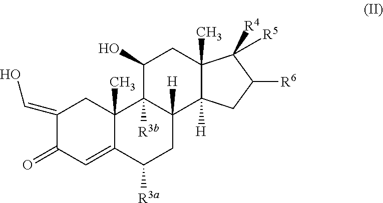
wherein R^{3a}, R^{3b}, R⁴, R⁵ and R⁶ are as defined in formula (I), with a compound of formula (III) or an acid addition salt thereof 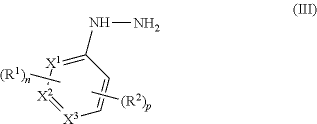
wherein n, p, R¹, R², X¹, X² and X³ are as defined in formula (I), and optionally thereafter carrying out one or more of the following procedures:
converting a compound of formula (I) into another compound of formula (I)
removing any protecting groups
forming a pharmaceutically acceptable salt.

12. A pharmaceutical composition comprising a compound of formula (I) as claimed in claim 1 or a pharmaceutically acceptable salt thereof in association with a pharmaceutically acceptable adjuvant, diluent or carrier.

13. A compound of formula (I) as claimed in claim 1 or a pharmaceutically acceptable salt thereof for use in treating asthma, chronic obstructive pulmonary disease or allergic rhinitis.

14. Use of a compound of formula (I) as claimed in claim 1 or a pharmaceutically acceptable salt thereof in the manufacture of a medicament for use in treating asthma, chronic obstructive pulmonary disease or allergic rhinitis.

15. A combination of a compound of formula (I) as claimed in claim 1 or a pharmaceutically acceptable salt thereof and one or more agents independently selected from:
a non-steroidal glucocorticoid receptor agonist;
a selective β₂ adrenoceptor agonist;
a phosphodiesterase inhibitor;
a protease inhibitor;
a glucocorticoid;
an anticholinergic agent;
a modulator of chemokine receptor function; and
an inhibitor of kinase function.
